(19) United States
(12) Patent Application Publication
(10) Pub. No.: US 2025/0213400 A1
(43) Pub. Date: Jul. 3, 2025

(54) ABSORBENT CORE HAVING CHANNELS, AND ABSORBENT ARTICLE WITH ABSORBENT CORE

(71) Applicant: Essity Hygiene and Health Aktiebolag, Göteborg (SE)

(72) Inventors: Lucas BÄCK, Göteborg (SE); Fanny STENHOLM, Göteborg (SE); Per BERGSTRÖM, Göteborg (SE); Montserrat MARTINEZ ESTELA, Puigpelat (ES)

(73) Assignee: Essity Hygiene and Health Aktiebolag, Göteborg (SE)

(21) Appl. No.: 18/841,756

(22) PCT Filed: Dec. 13, 2022
(86) PCT No.: PCT/EP2022/085622

§ 371 Date: Aug. 27, 2024

(30) Foreign Application Priority Data
Feb. 28, 2022 (WO) PCT/EP2022/054913
Feb. 28, 2022 (WO) PCT/EP2022/054991

(51) Int. Cl. A61F 13/537; A61F 13/53; A61F 13/534

(52) U.S. Cl. CPC A61F 13/53708; A61F 2013/530489; A61F 2013/530868; A61F 2013/53445

(57) ABSTRACT

An absorbent core having a length in a longitudinal direction and a width in a transverse direction, including upper and lower core cover layers and absorbent material sandwiched therebetween, and a pair of channels extending in the longitudinal direction in the absorbent material, each of the channels being free or substantially free from absorbent material and having a channel seal extending therein and joining the core cover layers. For at least one transversal segment of the absorbent core including the channel seals, one core cover layer has a first width between the channel seals, and the other core cover layer has a second width between the channel seals, wherein the first and second widths of the core cover layers are the widths of the core cover layers between the channel seals in an unfolded, flat-out state and wherein the first width is no less than 120% of the second width.

### TECHNICAL FIELD

The present disclosure pertains to an absorbent core comprising an upper core cover layer and a lower core cover layer and an absorbent material sandwiched between the upper core cover layer and the lower core cover layer. The absorbent article comprises a pair of channels in the absorbent material, each of the channels having a channel seal extending therein, the channel seal joining the upper and lower core cover layers within the channel. The disclosure further relates to an absorbent article comprising an absorbent core.

### BACKGROUND

In disposable absorbent articles, such as baby diapers and other articles for absorption of body fluids such as pant-type absorbent garments, incontinence protectors and sanitary napkins, there is a conflict between the requirement of sufficient absorption capacity and leakage security on the one hand and wearer comfort and discretion on the other hand. Accordingly, there is a demand for highly absorbent articles which are non-bulky and flexible, and which conform well to the body of a wearer. It is highly desirable that the wearer does not feel restricted or uncomfortable when wearing the absorbent article while still being confident that the article is efficient in preventing leakage. For adult user's it is particularly important that a pant-type garment resembles ordinary underwear as closely as possible and can be inconspicuously worn under normal tightfitting clothing. To provide sufficient absorption capacity while offering thin absorbent articles, a large proportion of the absorbent material may be what is commonly known as superabsorbent material or merely “superabsorbents”. The superabsorbents may be combined with absorbent fibers, predominantly cellulose pulp fibers, creating a fibrous network which contributes to distribute liquid in the absorbent core and to retain particulate superabsorbent material in the core. Superabsorbents are polymeric materials usually incorporated in absorbent articles in the form of fibers, particles, or granules. Superabsorbents can absorb many times their own weight of fluid upon swelling and formation of a hydrogel. Absorbent articles which contain a large amount of superabsorbent material have been found to lose their initial flexibility and wearer comfort as they absorb liquid and swell. High-impact areas of an absorbent article, such as the crotch portion of a diaper which contains a large amount of absorbent material distributed over a relatively small area may grow thick and become stiff as the article absorbs liquid. Moreover, it is a concern in disposable absorbent articles that the available absorption capacity of the absorbent material may be utilized as fully as possible, to keep material consumption at a minimum when producing the absorbent articles. It is therefore desirable that the full absorbent capacity of the superabsorbents can be utilized. In absorbent cores having a high content of superabsorbent material, such as 50% superabsorbent material or more and a relatively low amount of cellulose pulp fibers or no cellulose fibers, it has been proposed to provide additional stabilization of the absorbent core.

In WO2019/125231 A1 it has been suggested to stabilize an absorbent core and counteract sagging in the crotch portion of an absorbent article by forming two longitudinally extending channel sealings in the absorbent core. The absorbent core is enclosed within a core cover and the channel sealings join an upper side of the core cover with a lower side of the core cover and are arranged such that they divide the absorbent core into a center segment and two side segments. When the absorbent material in the absorbent core absorbs body fluids and swells, the core cover acts as swelling restriction means by limiting the swelling room for the absorption material in the different segments. Thereby, the expanding absorption material exerts outward pressure on the core cover resulting in an increase in stiffness of the absorbent core in the crotch portion of the absorbent article. Particularly high stiffness may thereby be achieved in the center segment of the absorbent core, between the two channel sealings.

### SUMMARY

The present disclosure is based on the insight that an absorbent article having improved fit, body conformance and wearer comfort as well as good absorbency and leakage security, may be achieved at least in part by the features of claim 1 or alternatively by the features of claim 10. Variations of the disclosure are set out in the dependent claims and in the following description.

The absorbent articles referred to herein may be wearable disposable absorbent articles, for example in the form of open diapers, pant diapers, belted diapers, incontinence garments, feminine hygiene garments and the like, as well as disposable absorbent inserts, e.g., incontinence shields or sanitary napkins, which are worn inside a support garment, such as a support pant or ordinary underwear. The articles are used to absorb, distribute, and store various types of body exudates while providing a high level of comfort and a sense of dryness to the wearer during wearing.

In a first aspect, the present disclosure is directed to an absorbent core having a length in a longitudinal direction and a width in a transverse direction, perpendicular to the longitudinal direction. The absorbent core comprises an upper core cover layer and a lower core cover layer and absorbent material sandwiched between the upper core cover layer and the lower core cover layer. The absorbent core comprises a pair of channels extending in the longitudinal direction in the absorbent material. Each of the channels is free or substantially free from absorbent material and has a channel seal extending therein, the channel seal joining the upper and lower core cover layers within the channel and extending along a longitudinal channel seal length. The absorbent core is divided into an imaginary set of one or more bordering transversal segments along the channel seal length, each transversal segment having a segment length of 10 mm and comprising the channel seals. The set comprises the maximum possible number of segments along the channel seal length. Further, the absorbent core is such that, for at least one transversal segment out of said set of segments, one out of the lower core cover layer and the upper core cover layer has a first width between the channel seals, and the other of the lower core cover layer and the upper core cover layer has a second width between the channel seals, wherein the first width is no less than 120% of the second width. The first and second widths of the core cover layers are the widths of the core cover layers between the channel seals in an unfolded, flat-out state.

During use of an absorbent article comprising an absorbent core as set out in the above, the absorbent material of the absorbent core will absorb body fluid and swell. The channel seals joining the upper core cover layer and the lower core cover layer in the channels will act as restriction means which limit swelling of the absorbent material in a transversal direction, as well as in a thickness direction by prohibiting the upper core cover layer and the lower core cover layer from freely moving away from the other. However, by one of out of the upper and lower core cover layers having a first width between the channel seals, and the other out of the upper and lower core cover layers having a second width between the channel seals, the first width being greater than the second width, additional space is provided between the channel seals for swelling of the part of the absorbent material entrapped between the channel seals and the upper and lower core cover layer. Thus, the absorbent core is provided with an improved swelling capacity and thereby improves utilization of the absorption capacity of the absorbent material.

The channel seals prevent separation of the upper and lower core cover layers and thereby restricts swelling of the absorbent material in the vicinity of the channels. The channel seals are preferably permanent seals, or at least a major part of each channel seal is a permanent seal which does not break under normal use and handling of the absorbent article. For example, the channel seals may be adhesive seals, weld seals or combinations of adhesive seals and weld seals.

The seal strength of the channel seals may for example be 2.5 N/25 millimeter or more, such as 3 N/25 millimeter or more, as measured according to the test method disclosed herein. There is no upper limit for the seal strength of the channel seal as the only requirement for a channel seal is that it does not break under the pressure which may be exerted on the seal during normal use.

It is to be understood that prior to the invention, in absorbent cores provided with channels having channel seals, the widths of the upper and lower core cover layers between the channel seals would be substantially equal. Hence, by providing a core cover with a first width between the channels of one of the core cover layers, and a second width between the channels of the other of the core cover layers, different from the first width, improved utilization of the absorption capacity of the absorbent material may be achieved without increasing the width between the channel seals of the absorbent core (as seen in a dry state of the absorbent core). The increased space for the absorbent material, as provided between the channel seals and delimited by the core cover layers, will provide for improved swelling capacity mainly in the thickness direction of the absorbent core.

Thus, absorbent cores with improved utilization of the absorption capacity may be achieved without increasing the width of the absorbent core (the mutual width of the upper and lower core cover layers), to the benefit of comfort and fit of the absorbent article comprising the absorbent core.

The first width is to be no less than 120% of the second width.

Optionally, in said at least one transversal segment, the first width may be no less than 130% of the second width. For example, the first width may be no less than 135% of the second width. For example, the first width may be in the range of from 120% to 160% of the second width, such as in the range of from 130% to 160% of the second width or in the range from 135% to 160%.

Optionally, in said at least one transversal segment, the first width differs from the second width by no less than 3 mm, such as by no less than 5 mm or by no less than 7 mm.

Optionally, in said at least one transversal segment, a third width as measured between the channel seals of the absorbent core is between 20 and 60 mm. For example, the third width may be between 25 and 45 mm.

The third width is to be measured on the absorbent core as such, in a dry state before use. (In other words, the third width is not to be measured in an unfolded state as the first and second widths.)

The absorbent material in said at least one transversal segment has an absorbent width. As such, the absorbent width may be measured from a first side edge to a second side edge of the absorbent material in the absorbent core.

In said at least one transversal segment, the upper core cover layer and the lower core cover layer extend over a mutual width being a core width as defined herein.

As such, a first longitudinal side edge and a second longitudinal side edge of the absorbent core are deemed to be longitudinal side edges delimiting said mutual width of the upper core cover layer and the lower core cover layer.

In some examples, in said at least one transversal segment, the upper core cover layer and the lower core cover layer may have the same width, in which case this width is the core width.

In other examples, in said at least one transversal segment, the upper core cover layer and the lower core cover layer may have different widths, in which case the mutual extensions of these different widths constitute the core width as defined herein.

The absorbent width may, in said at least one transversal segment, be less than the core width. As such, the upper and lower core cover layers have a mutual extension over the full absorbent width so as to enclose the absorbent material.

Optionally, at least in said at least one segment, the absorbent width may be substantially equal to the core width.

By virtue of the first width being greater than the second width, allowing for increased expansion of the absorbent material in the thickness direction of the absorbent core and between the channel joints, the core width may be selected to be relatively close to the absorbent width, while still providing an absorbent core with improved swelling capacity.

For example, at least in said at least one segment, the absorbent width may be no less than 90% of the core width, such as from 90% to 100% of the core width. For example, the absorbent width may be from 95% to 100% of the core width.

As mentioned in the above, the absorbent core has a third width in said at least one segment, the third width being measured between the channel seals of the absorbent core.

For example, the third width may be in the range of from 15% to 40% of the absorbent width, such as in the range from 20% to 35% of the absorbent width.

The two channels of the pair of channels may extend spaced apart and symmetrically arranged on each side of the longitudinal centre line of the absorbent material. As such, each channel may extend between the longitudinal centre line and a corresponding one of a first side edge or a second side edge of the absorbent material.

The channels may have different shapes. For example, the channels may be curved or the channels may be inclined in relation to the longitudinal direction. However, regardless of the shape, the channels will have an extension along the longitudinal direction. Similarly, the channel seals will have a channel seal length as seen along the longitudinal direction.

In some examples, the two channels of the pair of channels are straight and extend in parallel with the longitudinal direction.

Possibly, the absorbent core may comprise more than one pair of channels. Each pair of channels may for example extend over a separate longitudinal portion of the absorbent core.

For example, the length of each of the channels in the pair of channels may be in the range of from 50 millimeter to 500 millimeter. For example, the channel seal length of each of the channel seals may be in the range of from 50 mm to 500 mm.

For example, the length of each of the channels may be from 75 millimeter to 400 millimeter, from 100 millimeter to 300 millimeter or from 150 millimeter to 200 millimeter. For example, the channel seal length of each of the channel seals may be from 75 millimeter to 400 millimeter, from 100 millimeter to 300 millimeter or from 150 millimeter to 200 millimeter.

The core cover layers may be made by core over materials.

The basis weight of the core cover materials as used herein may for example be in the range of from 5 g/m² to 20 g/m².

The core cover material may be made of thermoplastic polymer material, such as polyolefin, polyesters, polyamide and combinations thereof. The core cover material may be a nonwoven material, a perforated plastic film, a netting, etc. A nonwoven layer may be formed by any of a variety of different processes, as known in the art, such as spunbonding, airlaying, meltblowing or bonded carded web formation processes. The nonwoven layer may be made of co-formed lamina of nonwoven materials such as an SMS (spunbond/meltblown/spunbond) nonwoven material an SMMS nonwoven material (spunbond/meltblown/meltblown/spunbond) or an SS (spunbond/spunbond) nonwoven material. The thermoplastic polymer materials in the nonwoven layer may be polypropylene or bicomponent fibers of polypropylene and polyethylene, or of a combination of such materials.

For example, the upper and lower core cover layers may be constituted by nonwoven materials.

The absorbent material may comprise or consist of cellulose pulp fibers and superabsorbent material.

The absorbent material may comprise superabsorbent material, preferably from 5% by weight to 80% by weight of the absorbent material is superabsorbent material. For example, the absorbent material may comprise from 30% by weight to 80% by weight of the superabsorbent material.

The absorbent material may comprise superabsorbent material, preferably in an amount of from 5% by weight to 80% by weight, such as from 30% by weight to 80% by weight, at least in a part of the absorbent core wherein the pair of channels is arranged. Accordingly, the superabsorbent content may for example be lower in a front end part and/or in a rear end part of the absorbent core than in a central part of the absorbent core, comprising the pair of channels.

The absorbent material may be constituted by one single absorbent component layer. Absorbent material comprising two or more layers are also contemplated for the absorbent articles as disclosed herein.

The superabsorbent material may be a polymeric material and may be in the form of particles, granules fibers, flakes, etc.

A relatively high amount of superabsorbent material in the absorbent core makes it possible to produce absorbent articles which are thin and discrete while offering high absorbent capacity and an ability of chemically binding absorbed fluid which results in an absorbent article having high leakage security and a dry inner surface even after having absorbed a large amount of fluid. The thin, superabsorbent-containing articles further has pre-use advantages, as they occupy less space for packaging, transport, and storage, as well as on a store shelf.

For example, the absorbent material between said channel seals may have a surface weight in the range of from 500 to 1200 gsm.

Optionally, the first and second core cover layers are formed from a single continuous cover material being wrapped around the absorbent material. When the core cover is formed from a single material layer, the single material layer may be wrapped around the absorbent core.

Optionally, the core cover may be formed by a separate upper core cover layer and a separate lower core cover layer.

The absorbent core may have any suitable shape in the plane defined by the longitudinal direction and the transverse direction. For example, the absorbent core may have an hourglass shape or a T-shape in the plane defined by the longitudinal direction and the transverse direction.

In another example, the absorbent core has a rectangular shape with side edges extending in the longitudinal direction and end edges extending in the transverse direction.

The absorbent core may comprise two or more parts having different absorption capacity. The absorbent core may be a three-dimensionally shaped, profiled core with a central part of the absorbent core being arranged between a first end part of the absorbent core and a second end part of the absorbent core, as seen in the longitudinal direction, the central part of the absorbent core having a greater thickness than a thickness of the first end part and a greater thickness than a thickness of the second end part.

The central part of the absorbent core may have a uniform first thickness, the first end part may have a uniform second thickness and the second end part may have a uniform third thickness. The first thickness may be equal to the second thickness. The central part of the absorbent core may be delimited from each of the first end part and the second end part by a corresponding first transition zone and a second transition zone.

The first end part may be a front end part and the first transition zone may be a front transition zone having an extension in the longitudinal direction of from 5 millimeter to 30 millimeter, such as from 10 millimeter to 20 millimeter, and wherein the second end part is a rear end part and the second transition zone is a rear transition zone having an extension in the longitudinal direction of from 20 millimeter to 80 millimeter, such as from 30 millimeter to 60 millimeter. The thickness of the absorbent core preferably changes linearly in the transition zones between the end parts and the central part.

A ratio between the thickness of the central part of the absorbent core and the thickness of the first end part may be in the range of from 4 to 1.5 and a ratio between the thickness of the central part of the absorbent core and the thickness of the second end part may be in the range of from 4 to 1.5.

The end parts may have equal thickness. However, end parts having different thickness are also conceivable for the absorbent articles as disclosed herein. A front end part may have a greater thickness than a rear end part.

The length of the absorbent core in an absorbent article as disclosed herein is determined as the distance between a point on the first end edge of the absorbent core where the first end edge intersects with the longitudinal center line and a point on the second end edge of the absorbent core where the second end edge intersects with the longitudinal center line, the length of the absorbent core being in the range of from 300 millimeter to 700 millimeter, such as from 350 millimeter to 600 millimeter or from 400 millimeter to 500 millimeter.

In an absorbent core having a thickened central part, the thickened central part may have a length in the order of from 30% to 70% of the length of the absorbent core, such as in the order of from 40% to 60% of the length of the absorbent core or from 45% to 55% of the length of the absorbent core. The length of the thickened central part may be approximately half the length of the absorbent core.

The absorbent core in an absorbent article as disclosed herein may have any planar shape, such as a rectangular shape, a T-shape, or an hourglass shape. Hourglass shapes include all configurations of the core where the end parts are wider than the central part. A planar shape with the central part being narrower than the end parts may be preferred over rectangular shapes, as a narrow central part may fit more comfortably in the crotch of a wearer. Moreover, a narrow central part of the absorbent core may allow greater transverse expansion of the core during use of the absorbent article without causing discomfort.

An absorbent core as disclosed herein, may comprise a three-dimensionally shaped core with a thicker central part and thinner end parts, the core being enclosed by a core cover comprising an upper layer and a lower layer. The upper and lower layers may be formed from a single web material or from two web materials. The cover material may be a nonwoven material. The core has a pair of sealed channels arranged therein, such as two sealed channels being symmetrically arranged on each side of a longitudinally extending center line of the article. The upper and lower layers of the core cover are bonded to each other with channel seals which are arranged in the channels. The upper and lower layers are in contact with each in side regions extending along the side edges of the absorbent core. The one or more channels are free from absorbent material, implying that the upper and lower layers of the core cover are directly bonded to each other in the channel seals. The absorbent core may also be shaped in the plane, such as having an hour-glass shape with the central part of the absorbent core being narrower than the first and second end parts.

In a three-dimensionally shaped absorbent core as disclosed herein, the thinner end parts may have a uniform or substantially uniform thickness, and the thicker central part may have a uniform or substantially uniform thickness. Transition zones may be arranged between the thicker central part and the thinner end parts, the thickness of the absorbent core preferably diminishing linearly or substantially linearly from the central part to the end parts within the transition zones. The end parts may be a designated front end part and a designated rear end part, with the rear end part having a greater extension in the longitudinal direction of the absorbent article than the front end part. The rear transition zone may have a greater extension in the longitudinal direction of the absorbent article than the front transition zone, as disclosed herein.

The pair of channels may be at least partly, such as completely arranged in the central part of the absorbent core.

Optionally, in at least a part of the absorbent core, the upper core cover layer and the lower core cover layer are connected in a first side seal in a first side region extending along a first side edge of the absorbent material and in a second side seal in a second side region extending along a second side edge of the absorbent material. Said part preferably comprises said at least one transversal segment.

The side seals may be permanent seals, i.e. intended to hold during the use of the absorbent core. However, in examples, a seal strength of the channel seals of the absorbent core may higher than a seal strength of each of the first and the second side seals of the absorbent core. Thus, the first and second side seals may be breakable seals. The channel seals prevent separation of the upper and lower core cover layers and thereby restricts swelling in the thickness direction of the absorbent core in the vicinity of the channels. The channel seals are preferably permanent seals, or at least a major part of each channel seal is a permanent seal which does not break under normal use and handling of the absorbent article. A permanent seal resists the forces arising from swelling absorbent material in the absorbent core and remains intact throughout use of the absorbent article. The side seals are breakable seals which break under influence of the forces exerted on the side seals as the absorbent material in the absorbent core absorbs fluid and expands. The breakable side seals together with the channel seals promote expansion of the absorbent material predominantly in the lateral or transverse direction of the absorbent core and the absorbent article.

For example, the seal strength of each one of the channel seals may be 2.5 N/25 millimeter or more, such as 3 N/25 millimeter or more and/or the seal strength of the first and the second side seals may be 2.0 N/25 millimeter or less, such as 1.5 N/25 millimeter or less, as measured according to the test method disclosed herein.

According to other examples, in at least a part of the absorbent core, the absorbent core is such that, in the portion of the core extending transversely beyond the first channel seal to a first side edge of the absorbent core, and in the portion of the core extending transversely beyond the second channel seal to a second side edge of the absorbent core, the upper core cover layer and the lower core cover layer are free from seals to each other. Thus, in this example, the absorbent core lacks seals connecting the upper core cover layer and the lower core cover layer in a first side region extending along a first side edge of the absorbent material and in a second side region extending along a second side edge of the absorbent material. The part of the absorbent core preferably comprises said at least one transversal segment.

As will be further explained in the below in relation to an absorbent article comprising such an absorbent core, the lack of seals between the upper core cover layer and the lower core cover layer outside the side edges of the absorbent material allows for swelling of the absorbent material in a respective transverse direction, outside of the core width, thus further improving the utilisation of the absorption capacity of the absorbent material.

Thus, an absorbent core as disclosed herein may comprise an upper core cover layer and a lower core cover layer sandwiching an absorbent material. The core may comprise a pair of sealed channels arranged therein, such as two sealed channels being symmetrically arranged on each side of a longitudinally extending center line of the article. The upper and lower layers of the core cover are bonded to each other with channel seals which are arranged in the one or more channels and which are extending along a longitudinal channel seal length. The pair of channels are free from absorbent material, implying that the upper and lower layers of the core cover are directly bonded to each other in the channel seals. The absorbent core is divided into an imaginary set of one or more bordering transversal segments along the channel seal length, each transversal segment having a segment length of 10 mm and comprising the channel seals. The set comprises the maximum possible number of bordering segments along the channel seal length.

The absorbent core is such that, for at least one transversal segment out of said set of segments, one out of the lower core cover layer and the upper core cover layer has a first width between the channel seals, and the other of the lower core cover layer and the upper core cover layer has a second width between the channel seals, wherein the first and second widths of the core cover layers are the widths of the core cover layers between the channel seals in an unfolded, flat-out state and wherein the first width is no less than 120% of the second width. The second width being greater than the first width implies that room for expansion of the absorbent material located transversally between the channels is provided, allowing for expansion of the absorbent material in between the channels primarily in the thickness direction of the absorbent core. The absorbent core may also be shaped in the plane, such as having an hour-glass shape with the central part of the absorbent core being narrower than the first and second end parts. Optionally, the portion of the core extending transversely beyond the first channel seal towards a first side edge, and in the portion of the core extending transversely beyond the second channel seal towards a second side edge, the upper core cover layer and the lower core cover layer may be free from seals to each other. The upper core cover layer and the lower core cover layer being free from seals to each other in said portions implies that the absorbent material located transversely outside of the pair of channels, on each side of the absorbent core, may extend in the respective transversal direction without being restricted by any such seals. For example, the absorbent material may expand in a transversal direction beyond the mutual extension of the upper and lower core cover layers. For example, the at least one transversal segment may comprise at least a quarter of the one or more segments in the set of segments, or at least half of the one or more segments in the set of segments. For example, the at least one transversal segment may comprise all of the segments in the set of segments.

An absorbent core as disclosed herein, may comprise a three-dimensionally shaped core material with a thicker central part and thinner end parts, the core material being sandwiched between an upper core cover layer and a lower core cover layer. The cover material may be a nonwoven material. The core comprises a pair of sealed channels arranged therein and which are extending along a longitudinal channel seal length, such as two sealed channels being symmetrically arranged on each side of a longitudinally extending center line of the article. The upper and lower layers of the core cover are bonded to each other with channel seals which are arranged in the one or more channels. The pair of channels are free from absorbent material, implying that the upper and lower layers of the core cover are directly bonded to each other in the channel seals. The absorbent core is divided into an imaginary set of one or more bordering transversal segments along the channel seal length, each transversal segment having a segment length of 10 mm and comprising the channel seals. The set comprises the maximum possible number of bordering segments along the channel seal length. The absorbent core is such that, for at least one transversal segment out of said set of segments, one out of the lower core cover layer and the upper core cover layer has a first width between the channel seals, and the other of the lower core cover layer and the upper core cover layer has a second width between the channel seals, wherein the first and second widths of the core cover layers are the widths of the core cover layers between the channel seals in an unfolded, flat-out state and wherein the first width is no less than 120% of the second width. For example, the at least one transversal segment may comprise at least a quarter of the one or more segments in the set of segments, or at least half of the one or more segments in the set of segments. For example, the at least one transversal segment may comprise all of the segments in the set of segments. The second width being greater than the first width implies that room for expansion of the absorbent material located transversally between the channels is provided, allowing for expansion of the absorbent material in between the channels primarily in the thickness direction of the absorbent core. The absorbent core may also be shaped in the plane, such as having an hour-glass shape with the central part of the absorbent core being narrower than the first and second end parts. Optionally, the portion of the core extending transversely beyond the first channel seal towards a first side edge, and in the portion of the core extending transversely beyond the second channel seal towards a second side edge, the upper core cover layer and the lower core cover layer may be free from seals to each other. The upper core cover layer and the lower core cover layer being free from seals to each other in said portions implies that the absorbent material located transversely outside of the pair of channels, on each side of the absorbent core, may extend in the respective transversal direction without being restricted by any such seals. For example, the absorbent material may expand in a transversal direction beyond the mutual extension of the upper and lower core cover layers.

In a three-dimensionally shaped absorbent core as disclosed herein, the thinner end parts may have a uniform or substantially uniform thickness, and the thicker central part may have a uniform or substantially uniform thickness. Transition zones may be arranged between the thicker central part and the thinner end parts, the thickness of the absorbent core preferably diminishing linearly or substantially linearly from the central part to the end parts within the transition zones. The end parts may be a designated front end part and a designated rear end part, with the rear end part having a greater extension in the longitudinal direction of the absorbent article than the front end part. The rear transition zone may have a greater extension in the longitudinal direction of the absorbent article than the front transition zone, as disclosed herein.

In a second aspect, there is provided an absorbent article comprising an absorbent core according to the first aspect.

The absorbent article may, in at least a part of the absorbent article, comprise a first edge seal extending along and directly adjacent a first side edge of the absorbent material and a second edge seal extending along and directly adjacent a second side edge of the absorbent material. The part of the absorbent article may preferably comprise said at least one segment.

With “directly adjacent” is meant that no other seal is present between the first or second side edge of the absorbent material, respectively, and the first or second edge seal. Thus, the first or second edge seal, respectively, will be the first seal which the absorbent material may reach if expanding in a transversal direction. The first and second edge seals thus form the seals limiting transversal expansion of the absorbent material at the first and second side edges thereof.

For example, where a sealed width is the transversal distance between the first edge seal and the second edge seal, and the upper core cover layer and the lower core cover layer of the absorbent core extend over a mutual width being a core width, the sealed width may be greater than the core width.

As already set out in the above, the upper core cover layer and the lower core cover layer extend over a mutual width being a core width as defined herein. In some examples, the upper core cover layer and the lower core cover layer may have the same width, in which case this width is the core width. In other examples, the upper core cover layer and the lower core cover layer may have different widths, in which case the mutual extensions of these different widths constitute the core width as defined herein.

The sealed width being greater than the core width thus implies that the first and second edge seals extend outside of the core width, i.e. outside of the core. In other words, the seals limiting transversal expansion of the absorbent material are located transversally outside of the core width. Thus, the absorbent material may expand outside of the core width in transversal directions. Accordingly, the absorbent article provides additional room for swelling of the absorbent material beyond a restricted core width.

Advantageously, in the absorbent article, the absorbent core may be such that the portion of the core extending transversely beyond the first channel seal towards a first side edge of the absorbent core, and in the portion of the core extending transversely beyond the second channel seal towards a second side edge of the absorbent core, the upper core cover layer and the lower core cover layer are free from seals to each other, as explained in the above. Thus, in this example, the absorbent core lacks side seals connecting the upper core cover layer and the lower core cover layer, and so the first and second edge seals extend directly adjacent the first and second side edges of the absorbent material, respectively.

That the absorbent core lacks side seals still implies that there may be some connection between the upper and lower core cover layers in the first and second side regions, although with very low integrity. For example, the upper and lower core cover layers may be kept together merely by frictional forces in the portions of the core cover extending laterally beyond the absorbent core.

The absorbent article may comprise one or more additional upper layers to the upper side of the upper core cover layer, and/or one or more additional lower layers to the lower side of the lower core cover layer.

For example, the first and second edge seals may be joining between one of said upper additional layer(s) and one of said lower additional layer(s).

For example, the first and second edge seals may be joining between one of said upper additional layer(s) and said lower core cover layer.

For example, the first and second edge seals are joining between one of said lower additional layer(s) and said upper core cover layer.

For example, one of said upper additional layer(s) may comprise a liquid permeable topsheet. The first and second edge seals may be joining between the topsheet and the lower core cover layer. In another example, the first and second edge seals may be joining between the topsheet and one of the lower additional layer(s).

For example, one of the lower additional layer(s) may comprise a liquid barrier layer. The first and second edge seals may be joining between the liquid barrier layer and the upper core cover layer. In another example, the first and second edge seals may be joining between the liquid barrier layer and one of the upper additional layer(s).

For example, the first and second edge seals may be joining between the topsheet and the liquid barrier layer.

As mentioned the above, the first and second edge seals may be permanent seals. That is, the seal strength of the first and second edge seals is such that it does not break under the pressure which may be exerted on the seal during normal use. As such, there is no upper limit for the seal strength of the edge seals.

As mentioned in the above, the channel seals are also to be permanent seals. However, since the pressure exerted on the channel seals during use may be greater than the pressure exerted on the first and second edge seals, the seal strength of the channel seals need not necessarily be the same as the seal strength of the edge seals.

For example, a seal strength of the channel seals may be, as exemplified in the above, 2.5 N/25 millimeter or more.

The seal strength of the edge seals may in some variants likewise be 2.5 N/25 mm or more. However, in other variants, the seal strength of the edge seals may be 1 N/25 mm or more.

For example, the edge seals and the channel seal(s) may be adhesive seals, weld seals or combinations of adhesive seals and weld seals. The seals may be formed as continuous bond lines or may be formed by discrete bond elements arranged along the seals.

Weld seals may comprise thermo-welded seals and other weld seals, such as ultrasonically welded seals. For weld seals such as e.g. thermo-welded seals and ultrasonically welded seals, the seal strength may be controlled by varying parameters such as sealing temperature, energy supply, sealing time and sealing pressure and/or by varying the sealing pattern.

For example, the first edge seal, the second edge seal and each channel seal may be adhesive seals.

In examples, the edge seals may be adhesive seals and the basis weight of adhesive in each edge seal may be 2 g/m2 or more, such as 3 g/m2 or more, or 5 g/m2 or more.

In an absorbent article as disclosed herein, the absorbent article may comprise a liquid permeable topsheet and a liquid barrier layer, the absorbent core being arranged between the liquid permeable topsheet and the liquid barrier layer. The liquid permeable topsheet and the liquid barrier layer are provided in addition to the upper core cover layer and the lower core cover layer. For example, the upper core cover layer may be facing the liquid permeable topsheet and the lower core cover layer may be facing the liquid barrier layer.

The liquid permeable topsheet, the liquid barrier layer and the absorbent core with the core cover may be part of or constitute an absorbent assembly or “core pack” which is produced as a separate unit and subsequently attached to front and back panels of an open-type or pant-type wearable article or to an outer cover of an open-type or pant-type wearable article.

As explained in the above, an article comprising the combination of an absorbent core, wherein, in at least one segment, one out of the lower core cover layer and the upper core cover layer has a first width between the channel seals, and the other of the lower core cover layer and the upper core cover layer has a second width between the channel seals, wherein the first and second widths of the core cover layers are the widths of the core cover layers between the channel seals in an unfolded, flat-out state and wherein the first width is no less than 120% of the second width, with and an article wherein a sealed width is the transversal distance between the first edge seal and the second edge seal, and the upper core cover layer and the lower core cover layer of the absorbent core extend over a mutual width being a core width, wherein the sealed width is greater than the core width, provides for additional space for swelling of the absorbent material over a predetermined core width. Preferably, the article may be such that the sealed width is greater than the core width for at least a part of the article comprising said at least one segment.

Thus, an additional space allowing for swelling of the absorbent material primarily in the thickness direction is provided between the pair of channels, and for swelling of the absorbent material primarily in the transversal directions is provided in the parts of the absorbent material located transversely outside of the pair of channels. Thus, the utilisation of the absorption capacity of the absorbent material in the article is improved.

However, the benefits of providing additional space for swelling of the absorbent material located transversely outside of the pair of channels may be achieved also without simultaneously providing for the additional space for swelling of the absorbent material in the thickness direction between the pair of channels.

Thus, in an alternative aspect, there is provided an absorbent article comprising an absorbent core, having a length in a longitudinal direction and a width in a transverse direction, perpendicular to the longitudinal direction. The absorbent core comprises an upper core cover layer and a lower core cover layer and absorbent material sandwiched between the upper core cover layer and the lower core cover layer, and comprising a pair of channels extending in the longitudinal direction in the absorbent material. Each of the one or more channels is free or substantially free from absorbent material and has a channel seal extending therein. The channel seal joins the upper and lower core cover layers within the channel and extends along a longitudinal channel seal length. The absorbent core is divided into an imaginary set of one or more bordering transversal segments along the channel seal length, each transversal segment having a segment length of 10 mm and comprising the channel seals. The set comprises the maximum possible number of bordering segments along the channel seal length. The absorbent core is such that, in at least a part of the absorbent article comprising at least one segment from the set of segments, the absorbent article comprises a first edge seal extending along and directly adjacent a first side edge of the absorbent material and a second edge seal extending along and directly adjacent a second side edge of the absorbent material, such that a sealed width is the transversal distance between the first edge seal and the second edge seal, and the upper core cover layer and the lower core cover layer of the absorbent core extend over a mutual width being a core width, and wherein the sealed width is greater than the core width.

Thus, in analogy with what is explained in the above, in the absorbent article according to the alternative aspect, the sealed width is greater than the core width which implies that the first and second edge seals extend outside of the core width, i.e. outside of the core. In other words, the seals limiting transversal expansion of the absorbent material are located transversally outside of the core width. Thus, the absorbent material may expand outside of the core width in transversal directions. Accordingly, the absorbent article provides additional room for swelling of the absorbent material beyond a restricted core width.

The absorbent article an accordance with the alternative aspect may be combined with any one of the features as set out in the above for the absorbent article of the second aspect, or for the absorbent core of the first aspect, providing the same benefits.

The absorbent core and/or the absorbent article as disclosed herein may be varied within the scope of the appended claims. For example, the materials and dimensions used for the different layers forming an absorbent core and/or absorbent article as disclosed herein may be varied, as indicated above. The absorbent article may further include any useful component or feature as known in the art such as fluid acquisition and distribution components, leg elastics, standing gathers, crotch and waist elastics, side panels, fastening systems, wetness indicators, skin care agents, disposal means, etc., as known in the art and depending on the type of absorbent article intended.

As set out in the above, some of the features are described as being applicable to at least one transversal segment of the set of segments comprising the channel seals. Some features are described as being applicable to at least one part of an article comprising at least one transversal segment of the set of segments comprising the channel seals.

Optionally, any such features may be applicable to at least a quarter of the segments in the set of segments, i.e the at least one transversal segment comprises at least a quarter of the segment in the set of segments.

Optionally, any such features may be applicable to at least half of the segments in the set of segments, i.e the at least one transversal segment comprises at least half of the segment in the set of segments.

Optionally, any such features may be applicable to all of the segments in the set of segments, i.e the at least one transversal segment comprises all of the segments in the set of segments.

As set out in the above, the set of segments may comprise one or more transversal segments. It will be understood that the number of segments in the set will be dependent on the longitudinal channel seal length, i.e. the length of the channel seals. Thus the number of segments in the set will correspond to the longitudinal channel seal length divided by 10 mm and rounded down to the closest integer. For example, the set of segments may comprise at least two transversal segments or at least three transversal segments. For example, the set of segments may comprise at least five transversal segments or at least 7 transversal segments.

With the term “width” as used herein is meant a maximum width as measured in the transversal direction of the absorbent core or article.

Width the term “length” as used herein is meant a maximum length as measured in the longitudinal direction of the absorbent core or article.

Unless explicitly said to be otherwise, widths and lengths are to be measured on the absorbent core or article as such, in a state as provided before use of a wearer.

With the term “thickness direction” is meant a direction perpendicular to the transverse direction and the longitudinal direction.

With the terms “upper” and “lower” as used herein are meant two opposed directions along the thickness dimension, i.e. perpendicular to the transverse direction and the longitudinal direction. Thus, the terms does not imply any specific orientation in a vertical direction. Further, the terms does not imply that in use, the absorbent core or article should have any particular direction visavi the user.

### BRIEF DESCRIPTION OF THE DRAWINGS

FIG. 1 shows a planar view of a stretched-out absorbent pant-type article with an absorbent assembly and with open side seams, as seen from an inner surface of the pant-type article;

FIG. 2 shows an enlarged view of the absorbent assembly in the pant-type article in FIG. 1 with an absorbent material enclosed in a core cover;

FIG. 3a shows a cross section through a version of the absorbent core in FIG. 2, taken along the line III-III when the absorbent core is in a dry state;

FIG. 3b shows a cross-section through another version of an absorbent core;

FIG. 4a shows a cross section through the absorbent assembly in FIG. 2, taken along the line III-III when the absorbent core is in a dry state;

FIG. 4b shows a cross section through the absorbent assembly in FIG. 2, taken along the line III-III when the absorbent core is in a wet state;

FIG. 5 shows a cross section through another variant of the absorbent assembly in FIG. 2, taken along the line III-III when the absorbent core is in a dry state;

FIG. 6 shows the pant-type article in FIG. 1 with the side seams closed and in a non-stretched out condition, as it appears before use;

FIG. 7 shows how the test pieces are mounted in the clamps of the test instrument, for performing the seal strength test method as described herein;

FIG. 8 shows how the test pieces are mounted in the clamps of the test instrument, for performing the seal strength test method as described herein;

FIG. 9 shows portions of a first core cover layer and a second core cover layer when in an unfolded state.

FIG. 10 shows how a test piece is mounted for performing width measurements in an unfolded state;

FIG. 11 shows a schematic view of a method as disclosed herein;

FIG. 12 shows a top view of a first core cover web being applied to a mat-forming surface in a method according to prior art;

FIG. 13 shows a cross section through the first core cover web and the mat-forming surface in FIG. 12, taken along the line III-III;

FIG. 14 shows a top view of a first core cover web being applied to a mat-forming surface in a method as disclosed herein; and

FIG. 15 shows a cross section through the first core cover web and the mat-forming surface in FIG. 14, taken along the line V-V.

### DETAILED DESCRIPTION

Different aspects of the present disclosure will be described more fully hereinafter with reference to the enclosed drawings. The embodiments disclosed herein can be realized in many different forms and should not be construed as being limited to the aspects set forth herein. It is to be understood that although the absorbent article shown in the Figures is a pant-type article, the absorbent core as described in the examples may be used in any disposable absorbent article for absorbing urine, feces and/or vaginal discharges, as disclosed herein.

The drawings are schematic and individual components, such as layers of material are not necessarily drawn to scale.

The pant-type article which is shown in the figures is a simplified article, and the article may contain further features, such as barrier cuffs. It is also to be understood that the waist elastic disclosed herein is optional or any other suitable type of waist elastic may be used. The side seams may be reclosable side seams, and the pant-type article may be provided with fastener elements to provide reclosability of the side seams.

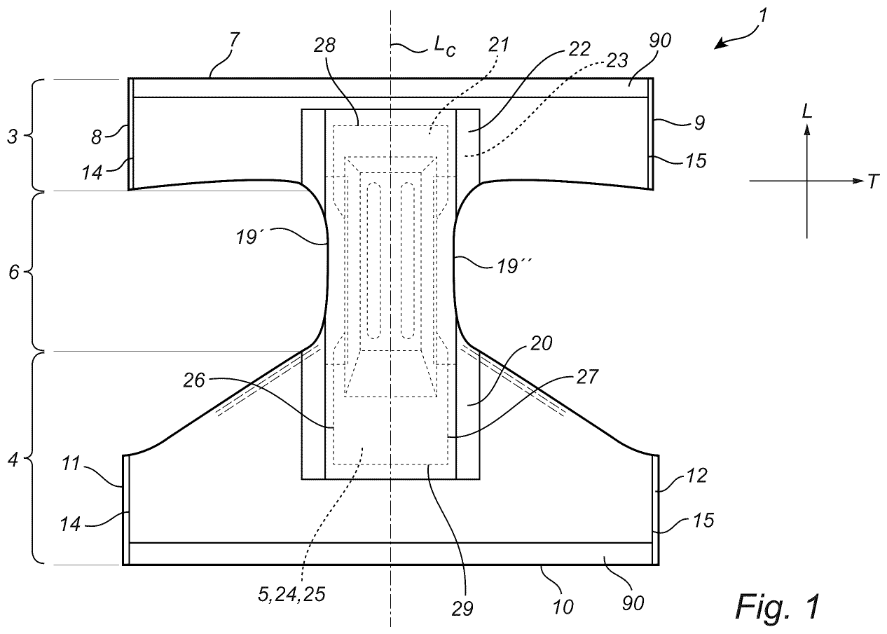

With initial reference to FIG. 1, there is shown a pant-type article 1 in the form of a pant-type incontinence article for adult users. The pant-type article 1 is shown in FIG. 1 in an unfolded and flat condition with all elastic elements in a fully extended state. The pant-type article has a longitudinal direction L and a transverse direction T, perpendicular to the longitudinal direction with a longitudinal centerline Lc extending in the longitudinal direction L.

The pant-type article 1 is seen from the inner surface which is the surface which will be facing a wearer's body when the article is being worn and which is opposite the outer, garment-facing surface of the pant-type article 1.

The pant-type article 1 comprises a front portion 3, a rear portion 4 and an absorbent assembly 20 located in a crotch portion 6 of the pant-type article 1 and extending in the longitudinal direction L forward in over the front portion 3 and rearward in over the rear portion 4. The absorbent assembly 20 extends in the longitudinal direction L from the front portion 3 through the crotch portion 6 and into the rear portion 4. The absorbent assembly 20 in the pant-type article shown in the figures is a separately produced component which comprises an absorbent core 21 comprising an absorbent material 70 which is sandwiched between an upper core cover layer 24 and a lower core cover layer 25 and which is further sandwiched between a liquid permeable topsheet 22 and a liquid barrier layer 23. The absorbent core 21 is arranged between the topsheet 22 and the liquid barrier layer 23 with the upper core cover layer 24 facing the liquid topsheet 22 and the lower core cover layer 25 facing the liquid barrier layer 23.

As disclosed herein, the provision of an absorbent core 21 is not limited to absorbent articles having the absorbent core applied to the article as a component of a pre-fabricated absorbent assembly which already comprises a topsheet and a liquid barrier layer.

The front portion 3 has a front waist edge 7 extending in the transverse direction T and a pair of front side edges 8,9 extending in the longitudinal direction L. The rear portion 4 has a rear waist edge 10 extending in the transverse direction T and a pair of rear side edges 11, 12 extending in the longitudinal direction L.

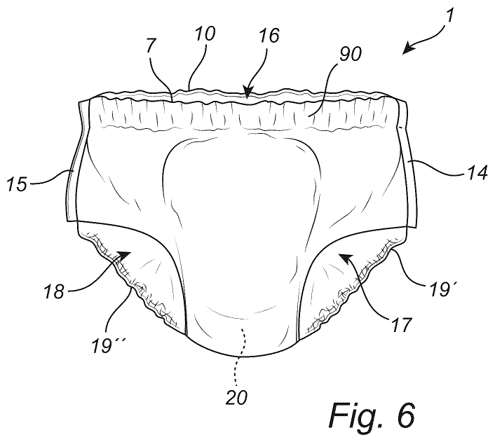

The first front side edge 8 is joined to the first rear side edge 11 in a first side seam 14 and the second front side edge 9 is joined to the second rear side edge 12 in a second side seam 15 to create the pant-type article 1 having a waist opening 16, a first leg opening 17 and a second leg opening 18, as shown in FIG. 6.

The side seams 14, 15 of the pant-type article 1 may be generally band-shaped joins which are formed by ultrasonic welding or thermowelding. To have sufficient strength to withstand the forces to which the pant-type article 1 is exposed during donning of the article and to allow sufficient production tolerances, the side seams commonly have a width in the order of 5 to 10 millimeters. It is also known to make side seams having a width less than 5 mm.

It is generally desired that a soiled pant-type article can be easily removed without having to pull the article down over the legs of a user. Therefore, the side seams are commonly made such that they are breakable by manual force to allow a user or a caregiver to pull apart the side seams before removing a soiled pant-type article.

The waist opening 16 is defined by the front panel waist edge 7 and the rear panel waist edge 10. A first leg edge 19′ defines the first leg opening 17 and a second leg edge 19″ defines the second leg opening 18.

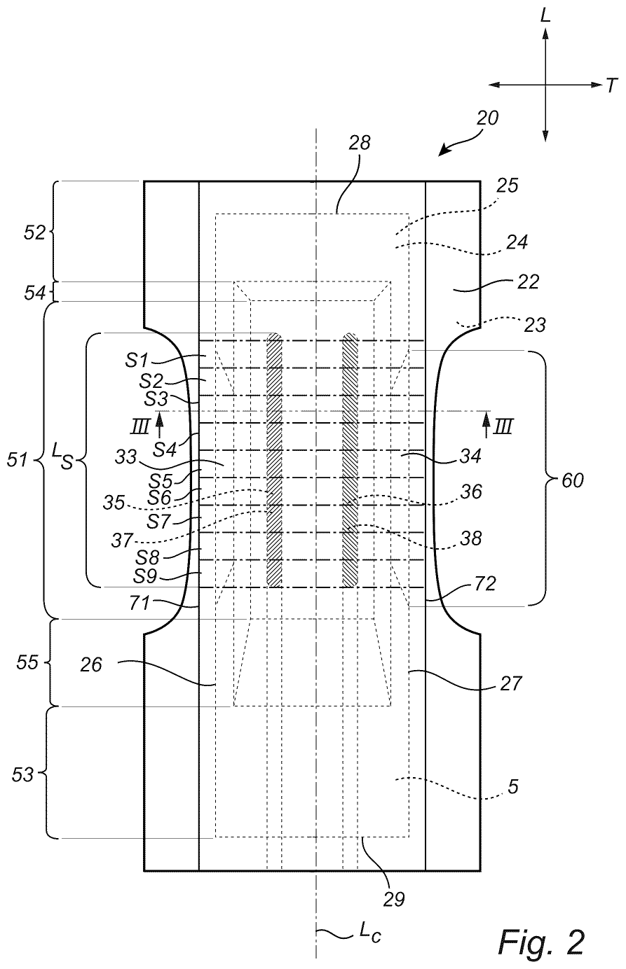

With reference to FIG. 2, the absorbent material 70 has an hourglass shape with a first side edge 26 and a second side edge 27, a first end edge 28 and a second end edge 29. The first side edge 26 and the second side edge 27 of the absorbent material 70 have a main extension in the longitudinal direction L and the first end edge 28 and the second end edge 29 have a main extension in the transverse direction T.

A first channel 35 and a second channel 36 are arranged in the absorbent material 70 and extend in the longitudinal direction L on either side of the longitudinal center line Lc and at a distance from each of the first and second side edges 26,27 of the absorbent material 70. The channels 35,36 are free or substantially free from absorbent material.

In addition, the upper and lower core cover layers 24, 25 are connected by a first channel seal 37 extending in the first channel 35 and by a second channel seal 38 extending in the second channel 36.

The upper and lower core cover layers 24, 25 may be formed from a single continuous cover material which is wrapped around the absorbent core 21. In the pant-type absorbent article which is shown in the figures, the upper and lower core cover layers 24,25 are formed from separate webs of material with the absorbent material 70 sandwiched between the core cover layers 24,25.

Depending on the type and size of absorbent article in which the one or more channels as disclosed herein are arranged, the length of the channels may range from 50 millimeter to 500 millimeter.

In a pant-type absorbent article for incontinent adult users, such as the absorbent article 1 shown in the figures, the channels 35,36 are preferably arranged in the crotch portion 6 of the article which is the narrow portion of the article which will be placed in the crotch of a user when the article is worn. The narrow crotch portion 6 of an absorbent article is the portion of the article which will receive a major part of excreted body fluid, such as urine. The crotch portion 6 may therefore have good absorption properties in terms of liquid acquisition, liquid distribution, and absorption capacity. The channels 35,36 in the absorbent material 70 contribute to rapid liquid acquisition and promote fluid distribution by channeling the fluid flow towards the front and rear portions 3, 4 of the absorbent article 1. In the absorbent article 1 which is shown in the figures, the length of the channels 35,36 substantially corresponds to the length of the crotch portion 6 and may typically be in the order of from 100 millimeter to 250 millimeter.

In wearable incontinence articles such as open diapers and pant-type diapers, the channels will typically have a length in the order of from 30% to 50% of the total length of the absorbent core. In smaller absorbent articles such as incontinence shields and sanitary napkins, the channels may extend almost to the ends of the absorbent core, such as up to 80% of the total length of the absorbent core.

In order to enhance the absorbent capacity in the narrow crotch portion 6 of the absorbent article 1, a central part 51 of the absorbent core 21 which is arranged between a first end part 52 of the absorbent core 21 and a second end part 53 of the absorbent core 21, as seen in the longitudinal direction L, has a greater thickness than the end parts 52,53. In the absorbent article 1 shown in the figures, the first end part 52 is the front end part 52 of the absorbent core 21 and the second end part 53 is the rear end part 53 of the absorbent core 21.

The central part 51 of the absorbent core 21 has a uniform thickness and each of the first end part 52 and the second end part 53 of the absorbent core 21 has a uniform thickness, the central part 51 of the absorbent core 21 being delimited from each of the first end part 52 and the second end part 53 by a corresponding first transition zone 54 and a second transition zone 55. The thickness of the absorbent core 21 diminishes linearly from the central part 51 of the absorbent core 21 to the end parts 52,53 of the absorbent core 21 within the transition zones 54,55. The rear end part 53 has a greater extension in the longitudinal direction of the absorbent article 1 than the front end part 52. In the absorbent core 21 shown in FIG. 2, the rear transition zone 55 has a greater extension in the longitudinal direction L of the absorbent article 1 than the front transition zone 54.

However, the transition zones may have an equal extension in the longitudinal direction of the absorbent article or the front transition zone may be longer than the rear transition zone. Absorbent cores with a thickened central portion and thinner end portions but without transition zones between the different portions of the cores are also contemplated for the absorbent articles as disclosed herein. Moreover, the central portion and the end portions may have non-uniform thickness.

The ratio between the thickness of the central part 51 of the absorbent core 21 and the thickness of the front end part 52 may be in the range of from 4 to 1.5 and the ratio between the thickness of the central part 51 of the absorbent core 21 and the thickness of the second end part 53 may be in the range of from 4 to 1.5. The thinner end parts 52, 53 may have the same thickness or may have different thicknesses.

Furthermore, it is to be understood that a three-dimensional shape of the absorbent core is optional to the absorbent articles as disclosed herein and that the absorbent core may be planar or substantially planar. In a planar or substantially planar absorbent core, the absorbent capacity in the absorbent core may be different in different parts of the core as a result of different amounts of superabsorbent material being arranged in the different parts of the core.

The absorbent core 21 as disclosed herein may comprise any absorbent material 70 suitable for absorbing discharged bodily wastes, such as cellulosic fluff pulp, tissue layers, highly absorbent polymers (super absorbents), absorbent foam materials including hydrogel-foam material, absorbent nonwoven materials, or the like. The absorbent core can comprise non-absorbent components such as stiffening elements, shaping elements, binders, etc. Various types of liquid-receiving and liquid distribution elements can also be included in the core.

The absorbent core 21 preferably comprises superabsorbent material in an amount of from 5% by weight to 80% by weight of superabsorbent material, such as from 30% by weight to 80% by weight of superabsorbent material, at least in the central part 51 of the absorbent core 21. As set out herein, the superabsorbent content may be lower in the front end part 52 and/or in the rear end part 53 of the absorbent core than in the central part 51 of the absorbent core 21 or may be the same in all parts of the absorbent core 21.

The superabsorbent material is a polymeric material and may be in the form of particles, granules fibers, flakes, etc.

As disclosed herein, the absorbent core 21 may comprise a mixture of absorbent cellulose fibers, such as cellulose pulp fibers, and superabsorbent material.

A high amount of superabsorbent material in the absorbent core, makes it possible to produce absorbent articles which are thin and discrete while offering high absorbent capacity and an ability to chemically bind and immobilize absorbed fluid which results in an absorbent article having high leakage security and a dry inner surface even after having absorbed a large amount of fluid.

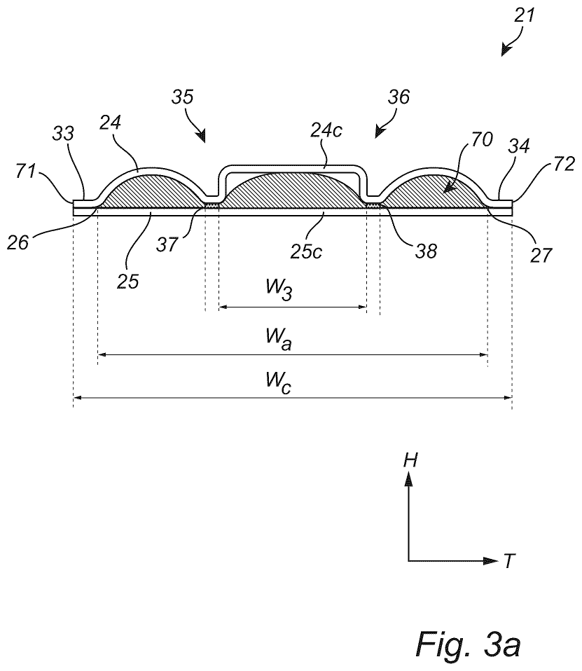

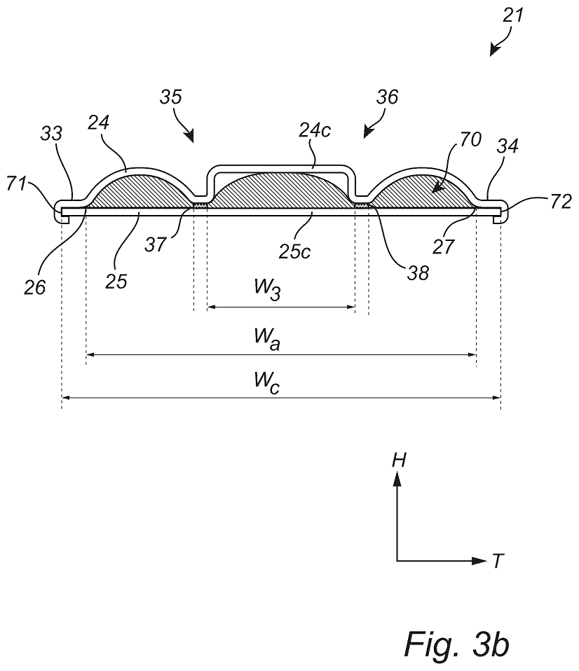

As schematically illustrated in FIG. 3, the absorbent core 21 may be divided into an imaginary set of one or more bordering transversal segments S1-S9 along the channel seal length Ls. Each transversal segment has a segment length of 10 mm and comprise a part of the channel seals. The set of one or more transversal segments comprises the maximum possible number of bordering segments along the channel seal length. As such, the number of segments in the set may be found by dividing the channel seal length Ls by 10 mm and rounding down to the closest integer.

In FIG. 2, an imaginary set of bordering transversal segments S1-S9 are illustrated by dash-dotted lines.

FIG. 3a shows a transversal cross section taken along the line III-III through the absorbent core 21 in FIG. 2. The depicted transversal cross-section is taken along a transversal centerline through one transversal segment S3 of the imaginary set of bordering transversal segments S1 to S9.

FIG. 3a shows the absorbent core 21 in a dry state, before the absorbent core 21 has absorbed any liquid.

The absorbent core has a length in the longitudinal direction L, a width in a transverse direction W, and a height in the thickness direction H, perpendicular to the longitudinal and transverse directions. The absorbent core 21 comprises an upper core cover layer 24 and a lower core cover layer 25, and absorbent material 70 sandwiched between the upper core cover layer and the lower core cover layer 25. The absorbent core 21 comprises a pair of channels 35, 36, extending in the longitudinal direction in the absorbent material 70, each of the channels 35, 36 being free or substantially free from absorbent material. Each of the channels 35, 36 has a channel seal 37, 38 extending therein, the channel seal 37, 38 joining the upper and lower core cover layers 24, 25 within the channel 35, 36. As seen in the transversal section of FIG. 3, the upper core cover layer 24 and the lower core cover layer 25 forms a space between the channel seals 37, 38, said space comprising absorbent material 70. In use, the absorbent material 70 will absorb liquid and swell. For example, when the absorbent material 70 comprises a superabsorbent material, the superabsorbent material will absorb liquid upon swelling and formation of an aqueous gel.

In order to fully utilize the absorbent capacity of the absorbent material 70, the absorbent article may provide sufficient expansion room for the superabsorbent material 70. As seen between the channel seals 35, 36, the room for swelling in a lateral direction is restricted.

As exemplified in FIG. 3a, to provide additional space for swelling of the absorbent material 70 between the channel seals 37, 38, the absorbent core 21 is such that the upper core cover layer 24 has a first width W1 as seen along the upper core cover layer 24 itself, i.e. in an unfolded flat-out state, and the lower core cover layer 25 has a second width W2 as seen along the lower core cover layer 25, i.e. in an unfolded flat-out state, where the first width W1 is greater than the second width W2. For example, the first width may be no less than 120% of the second width W2, such as no less than 130%. Thus, additional space for the absorbent material 70 between the channel seals 37, 38 is provided, allowing for increased swelling of the absorbent material 70 between the channels 35, 36 in the thickness direction. The first and second widths W1, W2 between the channel seals 37, 38 in an unfolded flat-out state may be determined in accordance with the method as described in the below method section of the application.

For example, the first width W1 may differ from the second width W2 by no less than 3 mm, such as no less than 5 mm or no less than 7 mm.

As exemplified in FIG. 3a, a third width may be measured between the channel seals 37, 38 core, this third width being in the range from 20 to 60 mm, such as in the range from 25 to 45 mm.

Alternatively, or in addition to providing additional space for swelling of the absorbent material 70 between the channel seals 37, 38, the absorbent core 21 may be adapted to provide additional space for swelling of the absorbent material 70 in the portions laterally outside of the channel seals 37, 38.

As illustrated in FIG. 3a, the upper core cover layer 24 and the lower core cover layer 25 may be in contact with each other in a first side region 33 extending along a first side edge 26 of the absorbent material, and in a second side region 34 extending along a second side edge 27 of the absorbent material 70.

As such, the first side region 33 is the region of the absorbent core 21 extending transversely beyond the first side edge 26 of the absorbent material 70 to a first side edge 71 of the absorbent core 21. Similarly, the second side region 34 is the region of the absorbent core 21 extending transversely beyond the second side edge 27 of the absorbent material 70 to a second side edge 72 of the absorbent core 21. As may be seen in FIG. 3a, the first and second side edges 71, 72 of the absorbent core 21 are deemed to be the edges delimiting the mutual extension of the upper core cover layer 24 and the lower core cover layer 25.

In some variants (not shown), the upper core cover layer and the lower core cover layer may be connected in a first side seal extending in the first side region 33 and a second side seal extending in the second side region 34.

To provide for additional space for swelling of the absorbent material 70 in the portions laterally outside of the channel seals 37, 38, i.e. laterally adjacent the first side region 33 and the second side region 34, respectively, the side seals may be adapted to be breakable seals, i.e. intended to break under influence of the forces exerted on the side seals as the absorbent material in the absorbent core absorbs fluid and expands. As such, the channel seals may have a seal strength being higher than a seal strength of each of the first and second side seals. To this end, for example, the seal strength of the channel seals may be 2.5/25 millimeter or more and the seal strength of the first and the second side seals may be 2.0 N/25 millimeter or less.

In other variants, such as exemplified in FIG. 2, the upper core cover layer 24 and the lower core cover layer 23 may be free from seals to each other. Thus, the first side region 33 extending along the first side edge 26 of the absorbent material, and the second side region 34 extending along the second side edge 27 of absorbent material may be free from seals.

The absorbent core 21 being free from seals in the first and second side regions 33,34 implies that there is no seal providing any significant seal strength. However, the first core cover layer 24 and the second core cover layer 25 could still have some slight connection, although with very low connection integrity, for example by frictional connection between the cover layers 24, 25.

When, as in the example of FIG. 3a, no side seals are present between the first core cover layer 24 and the second core cover layer 25, the absorbent material 70 in the portions transversely outside of the channels 35, 36 may thus swell in transverse directions, beyond the extension of the core width Wc, i.e. beyond the mutual extension of the core cover layers 35, 36.

As exemplified in FIG. 3a, the absorbent material 70 may have an absorbent width Wa as seen in the transversal cross-section of the core. Also in the transversal cross-section, the upper core cover layer 24 and the lower core cover layer 25 extend over a mutual width being the core width Wc.

The absorbent width Wa is less than the core width Wc. Due to the increased space for swelling of the absorbent material 70 which may be provided as explained in the above, for the absorbent material between the channel seals and/or for the absorbent material in the portions transversely outside of the channel seals, the core width Wc may be relatively narrow. As such, the absorbent width Wa may be no less than 90% of the core width Wc, such as no less than 95% of the core width Wc.

FIG. 3b illustrate another example of an absorbent core 21. The absorbent core 21 of FIG. 3b is similar to the absorbent core in FIG. 3a, but for that the upper core cover layer 24 is arranged to be folded around the transversal side edges of the lower core cover layer 25. The core width Wc being the mutual transversal extension of the upper core cover layer 24 and the lower core cover layer 25, is also in this case equal to the width of the lower core cover layer 25.

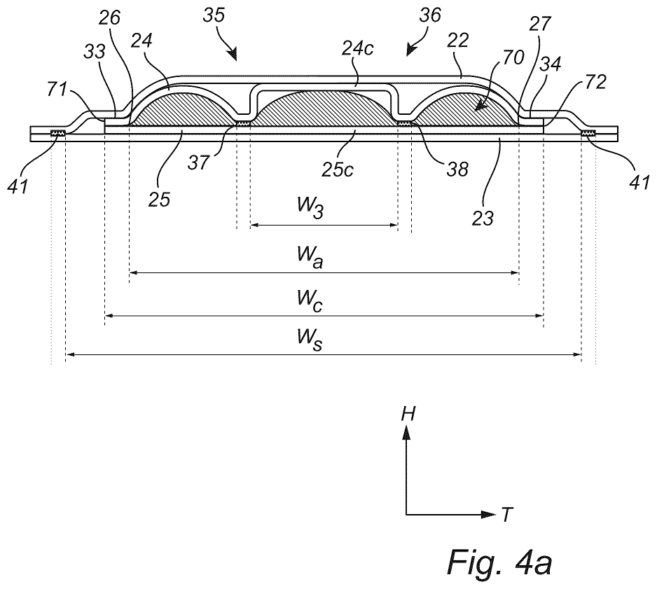

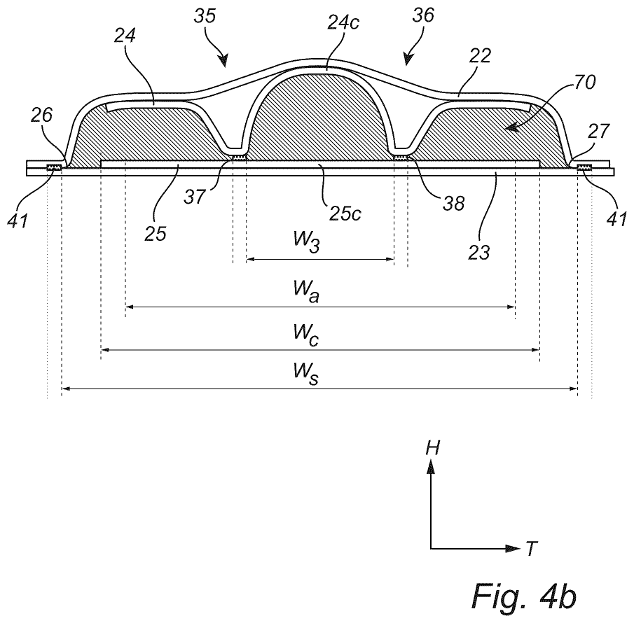

FIGS. 4a and 4b show a cross section taken along the line III-III through the absorbent assembly in FIG. 2, with an absorbent core 21 as exemplified in FIG. 3a. FIG. 4a shows the absorbent core 21 in a dry state, before the absorbent core 21 has absorbed any liquid and FIG. 4b shows the absorbent core 21 in a wet state, after absorption and swelling of the absorbent core 21.

The absorbent core 21 with the upper core cover layer 24 and the lower core cover layer 25 is shown as arranged between the liquid permeable topsheet 22 and the liquid barrier layer 23. The absorbent core 21 is arranged between the topsheet 22 and the liquid barrier layer 23 with the upper core cover layer 24 facing the liquid topsheet 22 and the lower core cover layer 25 facing the liquid barrier layer 23.

The topsheet 22 and the liquid barrier layer 23 extend beyond the periphery of the absorbent core 21 and are joined to each other in an edge seal 41 extending along the periphery of the absorbent core outward of the side edges 26, 27 and the end edges 28, 29 of the absorbent core 21. The edge seal 41 between the topsheet 22 and the liquid barrier layer 23 is preferably a permanent seal.

The upper core cover layer 24 and the lower core cover layer 25 extend over a mutual width in the absorbent core 21, the mutual width being a core width Wc as defined herein. In the example of FIGS. 3, 4a, the upper core cover layer 24 and the lower core cover layer 25 have the same width, thus being the core width Wc.

The edge seals 41 extend along and directly adjacent the first and second side edges 26, 27 of the absorbent material, respectively. By directly adjacent is meant that there is no other seal present between the first or second edge seal 41 and the first or second side edge of the absorbent material 70, respectively. Thus, the edge seals 41 will be the seals limiting the possible expansion of the absorbent material 70 in the transversal directions. The width between the first and second edge seals 41 is defined as a sealed width Ws.

In the illustrated transversal cross-section, the sealed width Ws is greater than the core width Wc. Hence, the first and second edge seals 41 extend transversely outside of the mutual extension of the upper and lower core cover layer 24, 25. This implies that the absorbent material 70 in the portions laterally outside of the channels 35, 36 may swell in transversal directions beyond the core width Wc. Thus, additional room for swelling is provided. It will be understood that this way of achieving additional space for swelling in transversal directions of the absorbent article may advantageously be combined with the above mentioned way of achieving additional space for swelling in the height direction of the absorbent article. Still, the two proposed ways of achieving additional space for swelling may also be used separately.

FIG. 4a illustrates, as mentioned in the above, a variant of the absorbent assembly when in a dry, unused state. FIG. 4b illustrates the absorbent assembly of FIG. 4a when the absorbent core has absorbed a relatively large amount of body fluid. As the absorbent core 21 absorbs body fluid, the absorbent material 70 in the absorbent core 21 swells, causing the portion of the absorbent material 70 which is located between the channels 35,36, and which is confined between the channel seals 37,38, to fill the available space between the channels 35, 36 and also to expand in the thickness direction. Thus, the additional space created by the first width W1 being greater than the second width W2 is utilized. The portions of the absorbent material 70 which are located laterally outside of the channels 35,36 with the permanent channel seals 37,38 may expand primarily in transversal directions into and beyond the side regions 33,34, until the side edges 26, 27 of the swelling absorbent material 70 reaches the edge seals 41. Thus, the lateral swelling of the absorbent material 70 may cause the side edges 26, 27 of the absorbent material 70 to move outward in the transverse direction T, whereby any hourglass shape (when initially present) of the absorbent material 70 may become gradually obliterated.

Other variants of the absorbent assembly are possible. For example, the absorbent assembly may comprise an absorbent core as exemplified in FIG. 3b.

Generally, the absorbent article may comprise one or more upper additional layers and/or one or more lower additional layers. One of said upper additional layer(s) may comprise a liquid permeable topsheet 22. One of said lower additional layer(s) may comprise a liquid barrier 23. The first and second edge seals 41, 42 may be joining between one of said upper additional layer(s) 22 and one of said lower additional layer(s) 23. For example, the first and second edge seals 41, 42 may be joining between one of said upper additional layer(s) 22 and said lower core cover layer 25. For example, the first and second edge seals 41, 42 are joining between one of said lower additional layer(s) 23 and said upper core cover layer 24.

As exemplified in FIGS. 4a-4b, the absorbent article may comprise a topsheet 22 adjacent the upper core cover layer 24 and a barrier layer 22 adjacent the lower core cover layer 25. As in the example of FIGS. 4a-4b, the edge seals 41 may join the topsheet 22 and the barrier layer 22.

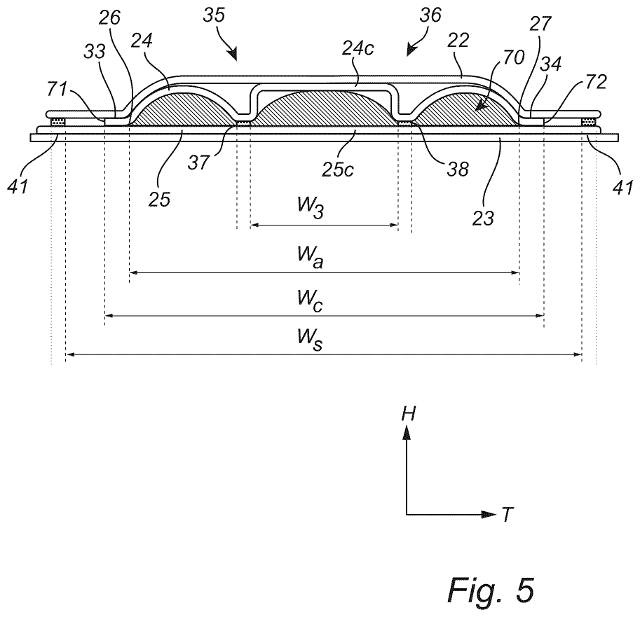

FIG. 5 illustrates another example of an absorbent article. FIG. 5 is similar to FIG. 4a, but for that the lower core cover layer 25 has a greater width than the upper core cover layer 24 (as seen in the dry state of the article). The core width Wc, which is the mutual width of the upper core cover layer 24 and the lower core cover layer 25, is thus in this case equal to the width of the upper core cover layer 24. The edge seals 41 is arranged so as to join the lower core cover layer 25 and the topsheet 22. As seen in FIG. 4, the sealed width Ws between the edge seals 41 is greater than the core width Wc.

The liquid permeable topsheet 22 may comprise or consist of a nonwoven material. Other suitable topsheet materials include tow fibers, porous foams, apertured plastic films and laminates and combinations of such materials. The materials which are best suited as topsheet materials are soft and non-irritating to the skin, are readily penetrated by body fluids, and display low rewet.

The liquid barrier layer 23 may consist of a thin plastic film, e.g. a polyethylene or polypropylene film, a nonwoven material coated with a liquid impervious material, a hydrophobic nonwoven material which resists liquid penetration or laminates of plastic film and nonwoven. The liquid barrier layer material may be breathable to allow vapour to escape from the absorbent body, while still preventing liquids from passing through the liquid barrier layer material.

The topsheet and/or the liquid barrier layer may further be attached to the core cover layers by any method known in the art, such as adhesive, heat-bonding, welding, etc.

The edge seals 41 may be formed for example by adhesive bonding, gluing or welding by heat or ultrasonically.

A pant-type articles as disclosed herein may have a two-part chassis with a crotch panel which is connected to a front panel along a front panel crotch edge, and which is connected to a rear panel along a rear panel crotch edge. The front and rear panels may be made from elastic or elasticized web material or an elastic laminate material, and the crotch panel may be a non-elastic web material or a non-elastic laminate material. Alternatively, the pant-type article may have a unitary chassis having a non-elastic outer or inner cover web extending the full distance between the front panel waist edge and the rear panel waist edge, the cover web constituting a non-elastic layer of each of the front panel and the rear panel and constituting the crotch web material in the crotch region of the pant-type article.

An elastically stretchable front body panel and an elastically stretchable rear body panel may comprise or consist of a stretchbonded laminated elastic web material.

Suitable stretch-bonded laminates may comprise nonwoven material layers or webs such as spunbond, air laid, wet laid, carded, electro spun or meltblown nonwovens. The nonwoven material may be bonded by any suitable technique, such as by needling, hydroentangling, ultrasonic welding, or thermobonding.

The fibers of the nonwoven materials used herein may be man-made fibers, natural fibers or mixtures of man-made and natural fibers. Man-made fibers include mono-component, bi-component and multicomponent fibers of polymers such as polyolefins, polyesters, polyacrylates, etc., as well as regenerated fibers such as viscose fibers and modal fibers. Natural fibers are for instance cellulosic fibers such as pulp fibers, cotton fibers, flax, hemp, etc.

The pant-type article 1 as disclosed herein may have an elastic waist feature 90 arranged along the waist opening 16. An elastic waist feature 90 may be formed by one or more elastic elements extending parallel with the front panel waist edge 7 and the rear panel waist edge 10. The elastic waist element or elements may be incorporated in the front portion 3 and the rear portion 4 or may be applied as a separate waistband which is attached to the front panel waist edge 7 and the rear panel waist edge 10. The pant-type article 1 which is shown in the Figures has an elastic waist feature 90 which extends around the full circumference of the waist opening 16. However, an elastic waist feature may be arranged only along a part of the waist opening, such as only along the rear waist edge, only along the front waist edge or along a part of one or both the front and the rear waist edge which part has a length which is less than the full length of the corresponding waist edge.

The elastic material in elastic elements arranged along the leg openings 17,18 and the waist opening 16 as disclosed herein may be any suitable elastic material such as natural or synthetic rubber, thermoplastic elastomers, such as thermoplastic polyurethane or styrene block co-polymers or elastane, also referred as to spandex (polyurethane-polyurea copolymer). The elastic elements may be of the elastane type that is available under the trade name “LYCRA”, but any suitable elastic thread may be used.

The nonwoven web-materials used in the absorbent articles as disclosed herein may comprise thermoplastic material. The nonwoven web-materials will typically be incorporated in joins and seams in the absorbent article and it may be desirable that the nonwoven webs be weldable by heat or by ultrasonic welding processes. Examples of suitable polymers for use in the fibrous nonwoven webs as disclosed herein are polyethylene polypropylene and other polyolefin homopolymers and copolymers and polyesters. The weldable nonwoven webs have a high content of thermoplastic component and preferably contain at least 50% thermoplastic fibers and more preferably at least 80% thermoplastic fibers.

### Manufacturing Method

For producing an absorbent core as described herein, suitable manufacturing methods may be used. For example, the absorbent may be produced by a method of manufacturing an absorbent core for a disposable absorbent hygiene article as disclosed herein comprises;
providing a first core cover web having a first surface and a second surface and advancing the first core cover web in a machine direction, the first core cover web having side edges extending in the machine direction, with a first core cover width being defined in a cross-machine direction between the side edges of the first core cover web, the cross machine direction being perpendicular to the machine direction;
reducing the width of the first core cover web, by introducing at least one pleat in the first core cover web, the at least one pleat extending in the machine direction between the side edges of the first core cover web and forming a pleated first core cover web having a second core cover width between the side edges of the pleated first core cover web, the second core cover width being smaller than the first core cover width;
applying an adhesive to the first surface of the pleated first core cover web;
advancing the pleated first core cover web onto a mat-forming surface of a mat-former, the mat-former having a recess arranged in the mat-forming surface, the recess having an air-permeable bottom surface and comprising at least one channel-forming element, the at least one channel-forming element being arranged on the bottom surface of the recess and protruding from the bottom surface of the recess;
bringing the pleated first core cover web into conformance with the recess and the at least one channel-forming element on the bottom surface of the recess, with the second surface of the first core cover web facing the bottom surface of the recess and causing the pleat in the first core cover web to at least partially unfold at the at least one channel-forming element;
applying absorbent material on the first surface of the first core cover web in the recess;
providing a second core cover web having a first surface and a second surface;
applying an adhesive on the first surface of the second core cover web;
applying the second core cover web to the first core cover web and to the absorbent material arranged on the first core cover web;
forming an absorbent laminate comprising the absorbent material, the second core cover web and the first core cover web, by pressing together the second core cover web; and
forming individual absorbent cores by severing the absorbent laminate in the cross-machine direction.

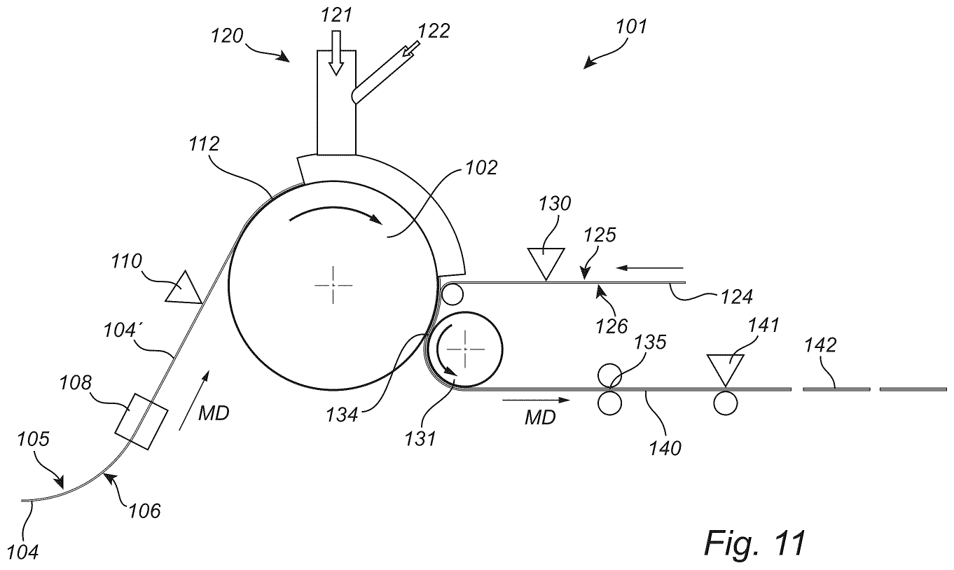

With initial reference to FIG. 11, there is shown a schematic representation of a method for producing absorbent cores for use in disposable absorbent hygiene articles.

The method which is shown in FIG. 11 is performed using a mat-former 101 comprising a rotating mat-forming drum 102. A first core cover web 104 having a first surface 105 and a second surface 106 is advanced in a machine direction MD.

The first core cover web 104 is passed through a pleat forming unit 108 where two pleats 109 are formed in the first core cover web 104, as seen in FIG. 14, the pleats 109 extending in the MD between the side edges of the first core cover web 104. Although two pleats are shown and described herein, it is to be understood that any suitable number of pleats may be formed in the first core cover web 104 by the pleat forming unit 108, such as one pleat, 3 pleats, or more. By pleating the first core cover web 104, the width w of the first core cover web 104 is reduced such that a second width 2 of the pleated first core cover web 104′ is smaller than the first width w1 of the non-pleated first core cover web 104. The widths w1, w2 of the non-pleated and pleated first core cover web 104, 104′ is measured between the side edges 111 of the first core cover web 104 in a cross machine direction CD perpendicular to the MD, as shown in FIG. 14.

In a next step, an adhesive, such as a liquid adhesive, is applied to the first surface 105 of the pleated first core cover web 104′ by a first adhesive applicator 110. After adhesive application, the pleated first core cover web 104′ is advanced onto a mat-forming surface 112 on the rotating mat-forming drum 102 of the mat-former 101. The mat-forming drum 102 has a recess 113 arranged in the mat-forming surface 112, see FIGS. 14 and 15, the recess 113 having a bottom surface 114 and comprising two channel-forming elements 115. The channel-forming elements 115 are arranged on the bottom surface 114 of the recess 113 and protrude from the bottom surface 114 of the recess 113. As for the pleats 109 in the first core cover web 104, the number of channel-forming elements at the bottom surface 114 of the recess 113 may be varied as disclosed herein. Accordingly, a single channel-forming element may be used, or more than two channel-forming elements may be used. The number of channel-forming elements 115 may correspond to the number of pleats 109 in the first core cover web 104 and the location of the pleats 109 in the first core cover web 104 may be synchronized with the placement of the channel-forming elements 115.

The pleated first core cover web 104′ is subsequently sucked into the recess 113 by a vacuum force V, which is applied from inside the rotating mat-forming drum 102 through the air permeable bottom surface 114 of the recess 113.

As is illustrated by FIG. 15, the pleated first core cover web 104′ as seen at the top of the figure, is moved into the recess 113, in the direction of the arrow A, and is brought to conform to the shape of the recess 113 and to the two channel-forming elements 115 in the recess 113, with the second surface 106 of the first core cover web facing the bottom surface 114 of the recess 113. During the process, the pleats 109 in the core cover web 104 are caused to unfold and cover the bottom 114 of the recess as well as the channel-forming elements 115, as seen in the lower portion of FIG. 15. The pleated core cover web 104′ is drawn into the recess 113 by the vacuum force V applied from inside the rotating mat-forming drum 102 and causing the pleats 109 in the pleated first core cover web 104′ to unfold at the channel-forming elements 115 and to readily conform to the shape of the channel-forming elements 115 due to the excess material which becomes available as the pleats 109 unfold. The pleats in the first core cover web 104 allows the first core cover web 104 to conform to the shape of the protruding channel-forming elements 115 without undue stretching or tensioning of the first core cover web 104.

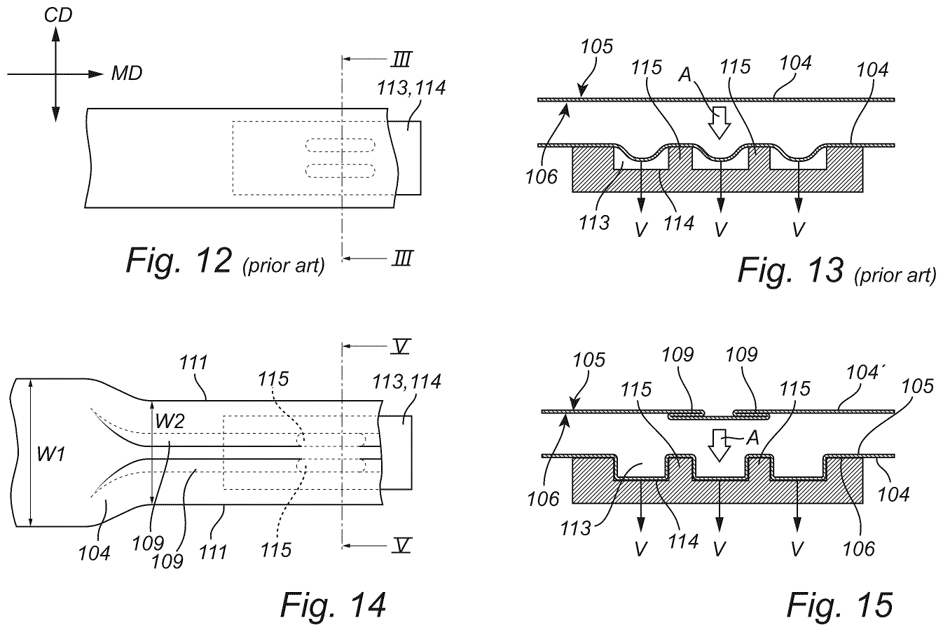

This differs from the prior art application of a first core cover web 104 to a recess 113 having channel-forming elements 115, as shown in FIGS. 12 and 13. In the prior art application of a first core cover web 104, the first core cover web 104 is sucked into the recess 113 by the vacuum force V, causing stress to build up in the material at the relatively distinct edges and corners of the channel-forming elements 115. The tension in the first core cover web material may give rise to tearing or piercing of the first core cover web. In addition, the prior art first core cover web 104 has a lower ability to conform to the shape of the recess 113, implying that the available space for filling absorbent material in the recess is reduced by non-conforming parts of the first core cover web 104, as is illustrated in FIG. 13.

Absorbent material is then applied on the first surface 105 of the first core cover web in the recess 113 on the mat-forming surface 112 of the rotating mat-forming drum 102. In FIG. 11, the application of absorbent material is illustrated by an air-laying unit 120 in which air-entrained wood pulp 121 is mixed with superabsorbent material 122, such as superabsorbent particles or fibres and the mixture is drawn into to the recess 113 which is clad with the first core cover web 104.

After application of the absorbent material, a second core cover web 124 having a first surface 125 and a second surface 126 is supplied and an adhesive, such as a liquid adhesive is applied to the first surface 125 of the second core cover web 124 by a second adhesive applicator 130. The second core cover web 124 is advanced onto a rotating wheel 131 with the second surface 126 of the second core cover web 124 facing the rotating wheel 131. The second core cover web 124 is applied to the mat-forming surface 112 of the rotating mat-forming drum 102 by the rotating wheel 131 and is brought to cover the first core cover web 104 and the absorbent material 121, 122 arranged on the first core cover web 104 in the recess 113.

The first surface 125 of the second core cover web 124 is attached to the first surface 105 of the first core cover web 104 and to the absorbent material 121, 122 arranged on the first core cover web 102 in the recess 113 in the mat-forming surface 112 of the mat-forming drum 102 by pressing together the second core cover web 124 and the first core cover web 104 in a nip 135 between two rollers, forming an absorbent laminate 140 comprising the absorbent material 121, 122, the second core cover web 124 and the first core cover web 104. Pressing together the second core cover web 124 and the first core cover web 104 brings the core cover webs in direct contact or substantially direct contact with each other in the generally absorbent material free areas created at the channel-forming elements 115 and in coinciding areas of the second core cover web 124 and the first core cover web 104 located outside the recess 113.

Alternatively, or in addition thereto, formation of the absorbent laminate 140 may involve pressing together the second core cover web and the first core cover web in the nip 134 between the rotating wheel 131 and rotating mat-forming drum 102.

After forming the absorbent laminate 140, individual absorbent cores 142 are formed by severing the absorbent laminate 140 in the cross-machine direction in a cutting unit 141.

Although the method as disclosed herein has been illustrated using a mat-former 101 with a rotating mat-forming drum 102, it is to be understood that the method as disclosed herein is equally applicable to mat-forming on a moving conveyor.

The recess 113 in the mat-forming surface 112 of the mat-forming drum 102 may be a continuous recess extending around the full circumference of the mat-forming drum 102 or the recess may be one of a plurality of recesses being equidistantly distributed around the circumference of the mat-forming drum 102.

As disclosed herein, each channel-forming element 115 in a continuous recess may consist of a plurality of element parts, the element parts being equidistantly distributed around the circumference of the mat-forming drum 102. When two channel-forming elements are arranged side-by side, the element parts are arranged in pairs around the circumference of the mat-forming drum 102. When severing the absorbent laminate 140 to form individual absorbent cores 142, the absorbent laminate 140 is severed in the cross-machine direction CD between the element parts. Accordingly, the number of channel-forming element parts of each channel-forming element 115 corresponds to the number of cores formed on the mat-forming drum during one revolution.

The adhesive may be uniformly distributed over the first surface 105 of the pleated first core cover web 104′. Likewise, the adhesive may be uniformly distributed over the first surface of the second core cover web 124.

Alternatively, the adhesive may be selectively distributed on one or both core cover web 104, 124 to create any desired bonding pattern.

Further details regarding a manufacturing method as set out in the above may be found in co-pending application PCT/EP2022/054991.

The disclosure may be varied within the scope of the appended claims. For example, materials and dimensions may vary.

### TEST METHODS

For all measurements, the article or the absorbent core is in a dry, unused state as provided before use.

### Test Method for Measuring Seal Strength

At least 10 days may pass between article manufacturing and the occasion for seal strength measurement.

The core with enclosing cover layers is carefully separated from other components of the absorbent article. If the lower core cover layer cannot be separated from the backsheet of the absorbent article without damaging the lower core cover layer, the backsheet should be left attached to the lower core cover layer when carrying out the seal strength test.

The exposed core with the core cover layers is placed flat on a support surface. If elastic is present in the core, the core should be stretched out and fixed in place on the support surface using strips of tape, mechanical fasteners, or similar.

Rectangular samples, 25 mm wide, are punched out from the core, each sample including a portion of the seal to be tested. The samples are punched out with the width direction of each sample parallel to the longitudinal centerline of the core and with the length direction of each sample perpendicular to the longitudinal centerline of the core. The samples are punched out such that the seal extends across an outer end of the sample, with inner end portions of the upper and lower core cover layers forming cover flaps extending towards the longitudinal centerline of the article. Any loose absorbent material between the core cover flaps is removed. The core cover flaps may be extended and/or reinforced with high friction tape, to ensure a stable and slip free attachment in the tensile tester clamps. It is of importance that the longitudinal edges of the test pieces are even and without break notches.

The samples are punched immediately adjacent one another. For example, from a seal having a length of 210 mm, eight 25 mm wide test samples may be punched out, with 10 mm of the seal remaining at one end of the seal and not being used for testing.

The samples, prepared as described above, are conditioned for 24 hours in a controlled environment with the temperature set to 23° C.+/−1° C. and 50%+/−5% relative humidity. Testing is performed in this same environment.

The samples are tested in a conventional tensile tester, such as available from Lloyd Instruments or the Instron Corporation.

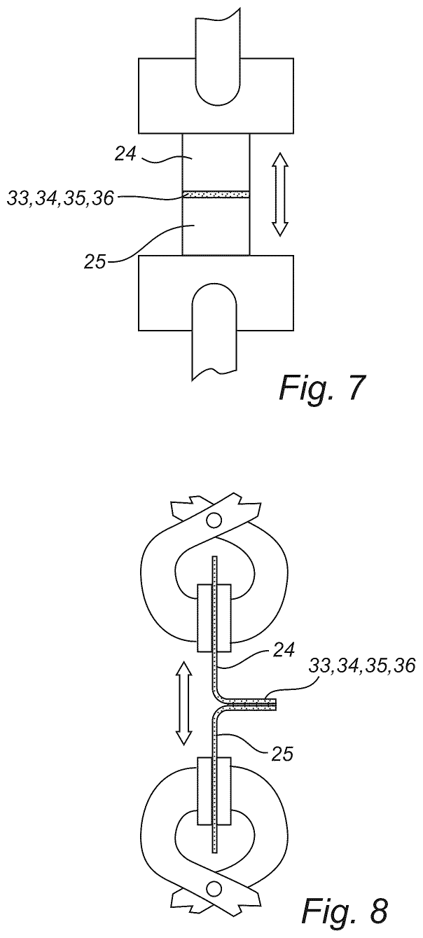

The core cover flaps are inserted vertically into the clamps of the tensile tester. The clamps should be as wide or wider than the sample. At insertion, tension over the seal should be avoided, but also excessive slack in the tested sample. The upper crosshead of the instrument is then set to move at a constant speed of 300 mm/min until the upper core cover layer separates from the lower core cover layer along the tested seal. The measured maximum force is registered in Newtons/25 mm (N/25 mm). FIGS. 7 and 8 illustrate how the test pieces are mounted in the clamps of the test instrument.

The seal strength of the tested seal is the arithmetic mean value based on all individual samples that can be cut along the part of the seal to be examined. For example, the strength of the entire 210 mm long seal as set out above, is the mean value of the seal strengths obtained from the testing of the eight test pieces which were punched out along the seal.

### Measurements of Thickness

Thickness is measured under a pressure of under a pressure of 0.5 kPa.

A suitable thickness gauge has a square foot measuring 5×5 cm (although smaller foots can be used for small regions of interest). The foot should be lowered gently onto the material under investigation, and a thickness value is read after 5 seconds.

### Measurements of Widths and Lengths

It is to be noted that with the terms “length” and “width” as used in this application is, unless otherwise stated, meant the greatest dimension in a longitudinal direction for length, and in a transverse direction for width, as seen on the absorbent core or article when in a dry unused state. Ahead of dimensional measurements, any elastic components are neutralized (such as by cutting them at regular intervals), and the object is placed flat against a planar surface. Measurements on the core are made after removal from any surrounding elastic chassis.

### Test Method for Measuring Widths of the Core Cover Layers in Folded Out State

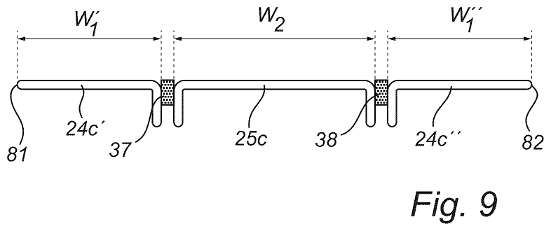

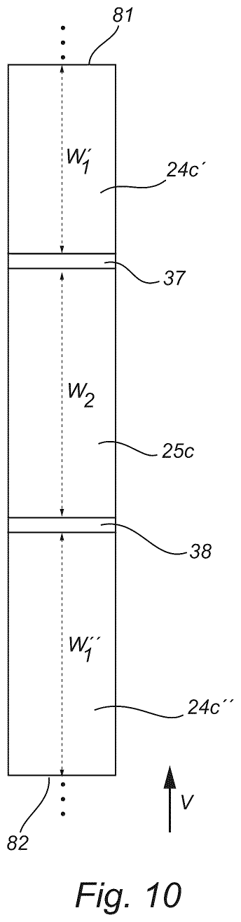

A method for measuring the widths of the core cover layers 24, 25 between the channel seals 37, 38 in an unfolded state and in a transversal segment of the absorbent core will now be described with reference to FIGS. 9 and 10.

The core with enclosing core cover layers 24, 25 is carefully separated from other components of the absorbent article. If the lower core cover layer 25 cannot be separated from the backsheet of the absorbent article without damaging the lower core cover layer, the backsheet should be left attached to the lower core cover layer when carrying out the width measurement.

The exposed core with the core cover layers is placed flat on a support surface. If elastic is present in the core, the core should be stretched out and fixed in place on the support surface using strips of tape, mechanical fasteners, or similar.

Rectangular samples, 10 mm wide, are punched out immediately adjacent one another from the core, each sample thus corresponds to a transversal segment in the imaginary set of bordering transversal segments S1-S9 of the absorbent core 21 as described in the above in relation to FIG. 2.

The samples are punched out with the width direction of each sample parallel to the longitudinal centerline of the core and with the length direction of each sample perpendicular to the longitudinal centerline of the core.

As mentioned in the above, the samples are punched immediately adjacent one another. For example, from an absorbent core with a pair of channels having channel seals where the longitudinal channel seal length is 215 mm, twenty-one 10 mm wide test samples may be punched out, with 5 mm of the channel seals remaining at one end and not being used for testing.

The samples, prepared as described above, are conditioned for 24 hours in a controlled environment with the temperature set to 23° C.+/−1° C. and 50%+/−5% relative humidity. Testing is performed in this same environment.

In each sample, a closed “tube” is formed by the portion of the upper core cover 24c extending between the channel seals 37, 38, and the portion of the lower core cover 25c extending between the channel seals 37, 38. The tube is filled with absorbent material 70.

The “tube” is cut open by cutting the upper core cover 24 along a line substantially parallel with the width direction of the sample (i.e. substantially parallel with the longitudinal axis L of the core) and in between the channel seals 37, 38. The absorbent material 70 is gently removed from the cut-open tube. The material lateral to the seals 37 can also be removed for the purpose of determining the widths of the core cover layers 24, 25 between the channel seals 37, 38 in an unfolded state.

The tube is gently unfolded. As such, the unfolded tube comprises, as seen in a length direction of the sample (transverse direction T of the core), a first cut edge 81 followed by a first portion 24c of the upper core cover 24, a first channel seal 37, a portion 25c of the lower core cover 25, a second channel seal 38, a second portion 24c′ of the upper core cover 24, ending with a second cut edge 82.

The channel seals 37, 38 are identified and their extensions along the length direction of the sample are marked.

The tube material is unfolded and hanged perpendicular to the longitudinal dimension of the sample to perform the measurements.

The first cut edge 81 is to extend along a horizontal direction. For example, the first cut edge 81 may be horizontally suspended by means of a clamp.

A weight of 10 g is attached to the second cut edge 82 of the tube material to stretch out the sample material in the length direction of the sample, such that the force from the weight is evenly distributed over the sample width. Thus, the sample material is suspended with the longitudinal direction of the sample parallel to a vertical direction.

The width W2 of the lower core cover material 25c between the channel seals 37, 38 is measured vertically between the markings identifying the channel seals 37, 38, and along the longitudinal direction of the sample. The measurement is made along the centreline of the sample.

The width W1 of the upper core cover material between the channel seals 37, 38 is the sum of the vertical length W1′ between the first cut edge 81 and the first channel seal 37, and the vertical length W1″ between the second channel seal 38 and the second cut edge 82. The measurements are made vertically and along the longitudinal direction of the sample. The measurements are made along the centreline of the sample.

Thus, the measurements of the widths W1, W2 between the channel seals 37, 38 in the sample are made at the same transversal cross-section for the first core cover layer and the second core cover layer.

From the measurements of the first and second widths W1, W2 in each sample (corresponding to one imaginary segment of the absorbent core), the relationship between the first and second width in the corresponding transversal segment may be calculated. Thus, the measurements made along the centreline of the sample are deemed to be representative for the corresponding transversal segment.

In some variants, each one out of the first and second widths W1, W2 may have the same value in each imaginary segment along the channel seal length Ls. This may be the case for example in some variants where the channels comprising the channel seals 37, 38 are straight and extend in parallel with the longitudinal direction.

In other variants, each one of the first and second widths W1, W2 may have different values in the different imaginary segments along the channel seal length. This may be the case for example in some variants where the channels comprising the channel seals 37, are straight but inclined in relation to the longitudinal direction L, or where the channels with the channel seals are curved.

Still, the relationship between the first width W1 and the second width W2 may be determined for each sample.

1. An absorbent core having a length in a longitudinal direction and a width in a transverse direction, perpendicular to the longitudinal direction, the absorbent core comprising an upper core cover layer and a lower core cover layer and absorbent material sandwiched between the upper core cover layer and the lower core cover layer, and comprising a pair of channels extending in the longitudinal direction in the absorbent material, each of the channels being free or substantially free from absorbent material and having a channel seal extending therein, the channel seal joining the upper and lower core cover layers within the channel and extending along longitudinal channel seal length, wherein
the absorbent core is divided into an imaginary set of one or more bordering transversal segments along the channel seal length, each transversal segment having a segment length of 10 mm and comprising the channel seals, and the set comprising the maximum possible number of bordering transversal segments along the channel seal length, wherein
the absorbent core is such that, for at least one transversal segment out of said set of segments,
one out of the lower core cover layer and the upper core cover layer has a first width between the channel seals, and the other of the lower core cover layer and the upper core cover layer has a second width between the channel seals, wherein the first and second widths of the core cover layers are the widths of the core cover layers between the channel seals in an unfolded, flat-out state, and wherein the first width is no less than 120% of the second width.

2. An absorbent core according to claim 1, wherein, in said at least one transversal segment, the first width is no less than 130% of the second width.

3. An absorbent core according to claim 1, wherein, in said at least one transversal segment, the first width differs from the second width by no less than 3 mm.

4. An absorbent core according to claim 1, wherein, in at least a part of the absorbent core, the upper core cover layer and the lower core cover layer are connected in a first side seal in a first side region extending along a first side edge of the absorbent material or in a second side seal in a second side region extending along a second side edge of the absorbent material, wherein a seal strength of each of the channel seals of the absorbent core is higher than a seal strength of each of the first or the second side seals of the absorbent core.

5. An absorbent core according to claim 1, wherein the seal strength of the channel seals is 2.5 N/25 millimeter or more or,
when in at least a part of the absorbent core, the upper core cover layer and the lower core cover layer are connected in a first side seal in a first side region extending along a first side edge of the absorbent material or in a second side seal in a second side region extending along a second side edge of the absorbent material, wherein a seal strength of each of the channel seals of the absorbent core is higher than a seal strength of each of the first or the second side seals of the absorbent core, the seal strength of the first and the second side seals is 2.0 N/25 millimeter or less.

6. An absorbent core according to claim 1, wherein, in at least a part of the absorbent core, the absorbent core is such that, in the portion of the core extending transversely beyond the first channel seal to a first side edge of the absorbent core, or in the portion of the core extending transversely beyond the second channel seal to a second side edge of the absorbent core, the upper core cover layer and the lower core cover layer are free from seals to each other.

7. An absorbent article comprising the absorbent core according to claim 1.

8. An absorbent article according to claim 7, wherein, in at least a part of the absorbent article, the absorbent article comprises a first edge seal extending along and directly adjacent a first side edge of the absorbent material or a second edge seal extending along and directly adjacent a second side edge of the absorbent material.

9. An absorbent article according to claim 8, wherein a sealed width is the transversal distance between the first edge seal and the second edge seal, and the upper core cover layer and the lower core cover layer of the absorbent core extend over a mutual width being a core width, wherein the sealed width is greater than the core width.

10. An absorbent article comprising an absorbent core, having a length in a longitudinal direction and a width in a transverse direction, perpendicular to the longitudinal direction, the absorbent core comprising an upper core cover layer and a lower core cover layer and absorbent material sandwiched between the upper core cover layer and the lower core cover layer, and comprising a pair of channels extending in the longitudinal direction in the absorbent material, each of the one or more channels being free or substantially free from absorbent material and having a channel seal extending therein, the channel seal joining the upper and lower core cover layers within the channel and having a longitudinal channel seal length, wherein,
the absorbent core is divided into an imaginary set of one or more bordering transversal segments along the channel seal length, each transversal segment having a segment length of 10 mm and comprising the channel seals, and the set comprising the maximum possible number of segments along the channel seal length,
wherein, in at least a part of the absorbent article comprising at least one segment from the set of segments,
the absorbent article comprises a first edge seal extending along and directly adjacent a first side edge of the absorbent material and a second edge seal extending along and directly adjacent a second side edge of the absorbent material, such that
a sealed width is the transversal distance between the first edge seal and the second edge seal, and the upper core cover layer and the lower core cover layer of the absorbent core extend over a mutual width being a core width, and wherein the sealed width is greater than the core width.

11. An absorbent article according to claim 10, wherein the article comprises one or more additional upper layers to the upper side of the upper core cover layer, and one or more additional lower layers to the lower side of the lower core cover layer, or
the first and second edge seals are joining between one of said upper additional layer(s) and one of said lower additional layer(s), or the first and second edge seals are joining between one of said upper additional layer(s) and said lower core cover layer, or the first and second edge seals are joining between one of said lower additional layer(s) and said upper core cover layer.

12. An absorbent article according to claim 11, wherein the one of said upper additional layer(s) comprises a liquid permeable topsheet.

13. An absorbent article according to claim 11, wherein the one of said lower additional layer(s) comprises a liquid barrier layer.

14. An absorbent article according to claim 10, wherein the edge seals and the channel seal(s) are adhesive seals, weld seals or combinations of adhesive seals and weld seals.

15. An absorbent article according to claim 14, wherein the first edge seal, the second edge seal and each channel seal are adhesive seals.

16. An absorbent core according to claim 1, wherein, in said at least one segment, a third width as measured between the channel seals of the absorbent core is between 20 and 60 mm.

17. An absorbent core according to claim 1, wherein, in said at least one segment, the absorbent material has an absorbent width, and the upper core cover layer and the lower core cover layer extend over a mutual width being a core width, wherein the absorbent width is equal to or less than the core width.

18. An absorbent core according to claim 17, wherein said absorbent width is no less than 90% of the core width.

19. An absorbent core according to claim 17, wherein said third width is in the range from 15% to 40% of said absorbent width.

20. An absorbent core according to claim 1, wherein the pair of channels are straight and extend in parallel with the longitudinal direction.

21. An absorbent core according to claim 1, wherein the length of each of the pair of channels is in the range of from 50 millimeter to 500 millimeter or the length of the channel seals is in the range of from 50 mm to 500 mm.

22. An absorbent core according to claim 1, wherein the upper and lower core cover layers are constituted by nonwoven materials.

23. An absorbent core according to claim 1 wherein the absorbent material comprises or consists of cellulose pulp fibers and superabsorbent material.

24. An absorbent core according to claim 23, wherein the superabsorbent material is present in the absorbent material in an amount of from 5% by weight to 80% by weight of the absorbent material.

25. An absorbent core according to claim 1, wherein the absorbent material between said channel seals has a basis weight in the range of from 500 to 1200 gsm.

26. An absorbent core according to claim 1, wherein the first and second core cover layers are formed from a single continuous cover material being wrapped around the absorbent material.

27. An absorbent core according to claim 1, wherein the upper core cover layer and the lower core cover layer are separate core cover layers.

28. An absorbent core according to claim 1, wherein the absorbent material has an hourglass shape or a T-shape in the plane defined by the longitudinal direction and the transverse direction.

29. An absorbent according to claim 1, wherein the absorbent material has a rectangular shape with side edges extending in the longitudinal direction and end edges extending in the transverse direction.

30. An absorbent core according to claim 1, the absorbent core comprising a central part being arranged between a first end part of the absorbent core and a second end part of the absorbent core, as seen in the longitudinal direction, the pair of channels being at least partly, arranged in the central part of the absorbent core.

31. An absorbent core according to claim 30, wherein the central part has a greater thickness than a thickness of the first end part and a greater thickness than a thickness of the second end part.

32. An absorbent core according to claim 31, wherein the central part of the absorbent core has a uniform thickness and each of the first end part and the second end part of the absorbent core has a uniform thickness, the central part of the absorbent core being delimited from each of the first end part and the second end part by a corresponding first transition zone and a second transition zone.

33. An absorbent core according to claim 32, wherein the first end part is a front end part and the first transition zone is a front transition zone having an extension in the longitudinal direction of from 5 millimeter to 30 millimeter and wherein the second end part is a rear end part and the second transition zone is a rear transition zone having an extension in the longitudinal direction of from 20 millimeter to 80 millimeter.

34. An absorbent core according to claim 32, wherein a ratio between the thickness of the central part of the absorbent core and the thickness of the first end part is in the range of from 4 to 1.5.

35. An absorbent core according to claim 32, wherein a ratio between the thickness of the central part of the absorbent core and the thickness of the second end part is in the range of from 4 to 1.5.

36. An absorbent core according to claim 32, wherein in the absorbent core at least two of the central part, the first end part, and the second end part have different absorption capacity.

37. An absorbent core according to claim 1, wherein the absorbent core has a length being determined as the distance between a point on a first end edge where the first end edge intersects with the longitudinal center line and a point on a second end edge where the second end edge intersects with the longitudinal center line, the length of the absorbent core being in the range of from 300 millimeter to 700 millimeter.

38. An absorbent core according to claim 1, wherein said at least one transversal segment of the absorbent core comprises at least a quarter of the segments in the set of segments.
